(19) United States
(12) Patent Application Publication
(10) Pub. No.: US 2005/0215503 A1
(43) Pub. Date: Sep. 29, 2005

(54) HIF oligonucleotide decoy molecules

(76) Inventors: Leslie M. McEvoy, Mountain View, CA (US); Lyn Powell, San Mateo, CA (US); Jie Zhang, Campbell, CA (US); Karen Morris, Los Altos, CA (US)

Correspondence Address: HELLER EHRMAN LLP, 275 MIDDLEFIELD ROAD, MENLO PARK, CA, 94025-3506, US

(21) Appl. No.: 11/003,907

(22) Filed: Dec. 2, 2004

Related U.S. Application Data
(60) Provisional application No. 60/526,869, filed on Dec. 3, 2003.
(60) Provisional application No. 60/612,406, filed on Sep. 22, 2004.

(51) Int. Cl. A61K 48/00; C12N 15/88; C07H 21/02

(52) U.S. Cl. 514/44; 435/458; 536/23.1

(57) ABSTRACT

The invention concerns double-stranded HIF decoy oligodeoxynucleotide (dsODN) molecules comprising a core sequence that is capable of specific binding to a HIF transcription factor, compositions containing such molecules, and their use in the treatment of various diseases and pathologic conditions associated with the regulation of gene transcription by a HIF transcription factor.

This application claims priority under 35 U.S.C. § 119(e)(1) of U.S. provisional patent application Ser. No. 60/526,869, filed on Dec. 3, 2003, and U.S. Provisional patent application Ser. No. 60/612,406, filed on Sep. 22, 2004, the entire disclosures of which are hereby expressly incorporated by reference.

### BACKGROUND OF THE INVENTION

1. Field of the Invention

The present invention concerns hypoxia-inducible factor (HIF) oligonucleotide decoy molecules and their use in the treatment of HIF-associated diseases or pathologic conditions.

2. Description of the Related Art

Hypoxia-inducible factor (HIF) is a heterodimeric transcription factor that mediates adaptive responses to changes in tissue oxygenation. Three subtypes of HIF are currently known (HIF-1, HIF-2, HIF-3); HIF-1 and HIF-2 have been shown to affect gene regulation via the conserved HRE. HIF-1 is a heterodimer that consists of a constitutively expressed HIF-1β subunit and a highly regulated HIF-1α subunit. The synthesis of HIF-1α is oxygen independent; however, the degradation is regulated primarily through oxygen-dependent mechanisms. Activated HIF-1α subunit migrates into the nucleus and dimerizes with the ARNT (aryl receptor nuclear translocator) subunit to form the active transcription factor HIF-1. HIF-1 recognizes the hypoxia-response element (HRE, or 5′-ACGTG-3′ (SEQ ID NO: 126) present in the enhancers or promoters of many genes and leads to their expression.

More than 60 putative direct HIF-1 target genes have been identified based on either the presence of a cis-acting hypoxia response element that contains a HIF-1 binding site, loss of hypoxia-induced expression of the genes HIF-1 α-null cells, or increased expression in von Hippel-Lindau (VHL) null cells, or in cells transfected with a HIF-1α expression vector.

Putative HIF-1 regulated genes include adrenomedullin, aldolase A, aldolase C, autocrine motility factor, cathepsin, endocrine gland-derived VEGF, endoglin, endothelin-1, erythropoietin (EPO), fibronectin 1, enolase 1, glucose transporter 1, glucose transporter 3, glyceraldehyde-3-P-dehydrogenase, hexokinase, insulin-like growth-factor 2, insulin-like growth-factor binding protein-1 and 2, keratin 14, 18, and 19, multidrug resistance 1, matrix matalloproteinase 2, nitric oxide synthase 2, plasminogen-activator inhibitor 1, pyruvate kinase M, transforming growth factor-α, transforming growth factor-β2, vascular endothelial growth factor (VEGF), urokinase plasminogen activator receptor, VEGF receptor-2 and vimentin (Semenza, Nature Rev. 3:721-732 (2003)).

Expression of some HIF-1 target genes, such as VEGF, is induced by hypoxia in most cell types, however, for the majority of HIF-1 target genes, expression is induced by hypoxia in a cell-type-specific manner.

Transcriptional induction by hypoxia was first identified and characterized in the 3′ flanking region of EPO by Beck et al., J. Biol. Chem. 266(24):15563-6 (1991), who defined regions responsible for the induction by transfection and mutagenesis. More specific nucleotide sequences responsible for responses to hypoxia were characterized and expanded to cells not expressing EPO by Semenza et al., J. Biol. Chem. 269(38):23757-63 (1994). These authors defined the sequence responsible for HIF induction as G/YACGTGC G/T (SEQ ID NO: 1) by functional analysis of the flanking sequences of three genes in the glycolytic pathway. This consensus sequence is accepted by subsequent authors as the canonical hypoxia responsive element (HRE). However, subsequent authors often define the HRE sequence more narrowly, as the 5 base pair core ACGTG (SEQ ID NO: 126), with various adjacent residues based on the analysis of their specific hypoxia regulated genes of interest. (See, e.g. Thornton et al. Biochem J. 350: Pt 1:307-12 (2000); and Miyazaki et al., J. Biol. Chem. 277(49):47014-21 (2002)).

HIF-1 activates the transcription of genes that are involved in crucial aspects of cancer biology, including angiogenesis, cell survival, glucose metabolism and invasion. Intratumoral hypoxia and genetic alterations can lead to HIF-1α subunit overexpression, which has been associated with increased patient mortality in several cancer types. HIF-1 and its pathway have been proposed as a target for development of anti-cancer agents (Semenza 2003, supra).

Double-stranded HIF-1 oligodeoxynucleotide decoy (dsODN) molecules have been used to investigate the biological role of HIF-1. HIF-1 dsODN molecules having the following sequences: 5′-GCCCTACGTGCTGTCTCA-3′ (sense) (SEQ ID NO: 128) and 5′-TGAGACAGCACGTAGGGC-3′ (antisense) (SEQ ID NO: 129) were described by Wang and Semenza, J. Biol. Chem. 268:21513-21518 (1993); Wang and Semenza, J. Biol. Chem. 270:1230-1237 (1995). HIF-1 decoy molecules were also disclosed in Oikawa et al., Biochem. Biophys. res. Commun. 289:39-43 (2001); and Yang and Zou, Am. J. Physiol. Renal Physiol. 281:F900-8 (2001).

### SUMMARY OF THE INVENTION

The present invention concerns double-stranded HIF decoy oligodeoxynucleotide (dsODN) molecules comprising a core sequence that is capable of specific binding to a HIF transcription factor, such as, for example, HIF-1 and/or HIF-2, compositions containing such molecules, and their use in the treatment of various diseases and pathologic conditions associated with the regulation of gene transcription by a HIF, e.g. HIF-1 and/or HIF-2 transcription factor.

In one aspect, the invention concerns dsODN molecules having a sense and an antisense strand, in which the sense strand comprises, in 5′ to 3′ direction, a sequence of formula FLANK1-CORE-FLANK2, wherein
CORE is the sequence ACTGT (SEQ ID NO: 126),
FLANK1, in which the nucleotide positions are designated by negative (−) numbers, is at least 6 nucleotides long, and
FLANK2, in which the nucleotide positions are designated by positive (+) numbers, has a GC content of at least about 50%, and
wherein said dsODN molecule is capable of specific binding to HIF.

In a specific embodiment, FLANK2 has a nucleotide other than G at position +1.

In another specific embodiment, FLANK2 has a nucleotide A at position +1.

In yet another embodiment, FLANK2 has a nucleotide A or G at position +3.

In a further embodiment, FLANK2 has any nucleotide at position +2.

In a still further embodiment, FLANK1 has a nucleotide other than A at position −1.

In a different embodiment, FLANK1 has a nucleotide T or C at position −1.

In another embodiment, FLANK1 has a nucleotide other than G at position −3.

In yet another embodiment, FLANK 1 has the nucleotide T at position −3.

In an additional embodiment, FLANK1 has the nucleotide G at position −4.

In a further embodiments, FLANK1 is at least 6, or at least 7 nucleotides long.

In a still further embodiment, the FLANK1-CORE-FLANK2 sequence is at least 14, or at least 16, or 14 to 28, or 16 to 24, nucleotides long.

In all embodiments, one or both strands may have a modified backbone and/or may comprise modified nucleotides.

In futher specific embodiments, the FLANK1-CORE-FLANK2 sequences are selected from the sequences listed in Tables 2A and 2B, sequnces with better binding properties being preferred.

Particularly included herein are FLANK1-CORE-FLANK2 sequences selected from the group consisting of decoy sequence Nos. 893 (SEQ ID NO: 161), 895 (SEQ ID NO: 162), 985 (SEQ ID NO: 207), 987 (SEQ ID NO: 208), 963 (SEQ ID NO: 196), 993 (SEQ ID NO: 211), and 995 (SEQ ID NO: 212).

While certain positions, substitutions and other variables are listed separately, it will be understood that any and all combinations of such variables are specifically covered. Thus, for example, dsODN decoy molecules comprising any combination of the listed nucleotides within the FLANK1 and FLANK2 sequences, in combination with any CORE sequence, are specifically within the scope of the invention.

In another aspect, the invention concerns method for modulating the transcription of a gene that is regulated by a HIF, such as a HIF-1 and/or HIF-2, transcription factor, comprising introducing into the nucleus of a cell containing such gene a HIF dsODN molecule of the invention.

In a further aspect, the invention concerns a method for the prevention or treatment in a mammalian host of a disease or condition associated with HIF-regulated gene transcription, comprising introducing into the cells of the mammal in vivo or ex vivo an effective amount of a double-stranded HIF decoy oligodeoxynucleotide (dsODN) molecule comprising a core sequence that is capable of specific binding to a HIF transcription factor, such as HIF-1 and/or HIF-2.

In a still further aspect, the invention concerns compositions, such as pharmaceutical compositions, comprising HIF dsODN molecules of the invention.

Specific diseases and conditions that are targeted by the dsODN molecules herein include, without limitation, cancer, inflammatory diseases, diseases including hypoxia in their pathology, cardiovascular diseases, stroke, diabetic retinopathy, Age-related Macular Degeneration, corneal neovascularization, conditions associated with pathogenic blood vessel growth, musculosceletal disorders, and other diseases and conditions the pathology of which involves HIF-activated gene transcription.

### BRIEF DESCRIPTION OF THE DRAWINGS

FIG. 1 is a matrix that computationally describes the base composition for both the core and the immediate-flanking regions of HIF decoy sequences of the invention.

FIG. 2 shows HIF decoy molecules of the invention, sorted by their binding affinity, highlighting certain shared sequences correlating with binding affinity.

FIG. 3 shows that a representative HIF decoy of the invention is a potent inhibitor of HIF activity.

FIG. 4 shows that a HIF decoy is able to complete with the immobilized EPO promoter binding site for HIF binding in nuclear cell extracts.

FIG. 5 shows that HIF decoy does not inhibit other transcription factors.

FIG. 6 shows that HIF decoy effectively competes for binding to the HIFα/HIFβ complex with the EPO and transferrin receptor promoters.

FIG. 7 shows that the binding of unrelated transcription factor, Oct-1, to its specific binding site is not inhibited by the HIF decoy.

FIGS. 8A and B show that HIFα activity, measured by gel shift (A), and secreted VEGF, measured by ELISA (B) were increased in the tested cell lines by hypoxia.

FIG. 9 shows that HIF decoy blocks HIF-1 activity in small cell lung cancer (SCLC), colon and pancreatic cancer cell lines.

FIG. 10 shows that low dose HIF decoy inhibits the growth of HT-29 colon tumor cell line.

FIG. 11 shows that HIF decoy induces apoptosis in the HT-29 colon tumor cell line.

FIG. 12 shows that HIF decoy reduces VEGF levels in the HT-29 colon tumor cell line.

FIG. 13 shows the efficacy of an HIF decoy in inhibiting growth of HT-29 colon tumor cell line relative to and in combination with Avastin™ (bevacizumab, Genentech, Inc.).

FIG. 14 shows that HIF decoy inhibits SCLC tumor growth and serum mVEGF levels.

FIG. 15 demonstrates dose-dependent efficacy of a HIF decoy in pancreatic xenografts.

FIG. 16 shows that HIF decoy induces apoptosis in the MiaPaCa pancreatic tumor cell line.

FIG. 17 is a sensitivity plot, displaying the effect of various nucleotide base substitutions at positions −4 through +3 of the sense strand on the binding affinity of a HIF oligonucleotide decoy molecule.

FIG. 18 illustrates the relationship between the predicted binding and observed competition ratio.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The term “double-stranded” is used to refer to a nucleic acid molecule comprising two complementary nucleotide strands connected to each other solely by Watson-Crick base pairing. The term specifically includes molecules which, in addition to the double-stranded region formed by the two complementary strands, comprise single-stranded overhang(s).

The terms “oligonucleotide decoy,” “double-stranded oligonucleotide decoy,” “oligodeoxynucleotide decoy,” and “double-stranded oligodeoxynucleotide decoy” are used interchangeably, and refer to short nucleic acid molecules comprising a double-stranded region, which bind to and interfere with a biological function of a targeted transcription factor. Accordingly, the terms “HIF oligonucleotide decoy,” “double-stranded HIF oligonucleotide decoy,” “HIF oligodeoxynucleotide decoy,” and “double-stranded HIF oligodeoxynucleotide decoy” are used interchangeably, and refer to short nucleic acid molecules comprising a double-stranded region, which bind to and interfere with a biological function of a HIF transcription factor. The term “double-stranded” is used to refer to a nucleic acid molecule comprising two complementary nucleotide strands connected to each other by Watson-Crick base pairing. The term “HIF decoy” and its synonyms specifically include HIF-1 and HIF-2 oligodeoxynucleotide decoy molecules. All HIF decoys, including HIF-1 and HIF-2 decoys, specifically include decoy molcules that, in addition to the double-stranded region formed by the two complementary strands, comprise single-stranded overhang(s). In addition, the term specifically includes HIF oligodeoxynucleotide decoy molecules in which, in addition to the double-stranded region, the two strands are covalently linked to each other at their 3′ and/or 5′ end.

The term “HIF-1” is used herein in the broadest sense and includes all naturally occurring HIF molecules of any animal species, including the HIF-1α/HIF-1β heterodimer and subunits thereof.

The term “transcription factor binding sequence” is a short nucleotide sequence to which a transcription factor binds. The term specifically includes naturally occurring binding sequences typically found in the regulatory regions of genes the transcription of which is regulated by one or more transcription factors. The term further includes artificial (synthetic) sequences, which do not occur in nature but are capable of competitively inhibiting the binding of the transcription factor to a binding site in an endogenous gene.

The term “binding affinity” refers to how tightly a given transcription factor will bind to a corresponding oligonucleotide decoy, which can be measured by various experimental approaches, including electromobility shift assays (EMSA) or TransAm assays, all described below.

The term “competition ratio” is the ability of a test decoy sequence to compete with a defined sequence for binding and retention of the transcription factor when compared to the defined sequence competing with itself in the TransAm assay (described in the examples). For example, if sequence A is bound to the TransAm plate, the competition ratio for Sequence B equals the absorbance of a well containing competitive sequence B divided by the absorbance of a well containing the competitive sequence. A smaller ratio refers to a higher competition ability to bind the transcription factor.

The term “specific binding” is used herein to mean that a particular decoy molecule binds to its target transcription factor, and does not significantly bind to any other transcription factor. In the case of HIF decoy molecules, specific binding allows for a decoy to bind more than one members of the HIF family, such as, for example, HIF-1 and HIF-2, but the decoys should not significantly bind to transcription factors which are not members of the HIF family.

As used herein, the phrase “modified nucleotide” refers to nucleotides or nucleotide triphosphates that differ in composition and/or structure from natural nucleotides and nucleotide triphosphates.

As used herein, the terms “five prime” or “5′” and “three-prime” or “3′” refer to a specific orientation as related to a nucleic acid. Nucleic acids have a distinct chemical orientation such that their two ends are distinguished as either five-prime (5′) or three-prime (3′). The 3′ end of a nucleic acid contains a free hydroxyl group attached to the 3′ carbon of the terminal pentose sugar. The 5′ end of a nucleic acid contains a free hydroxyl or phosphate group attached to the 5′ carbon of the terminal pentose sugar.

As used herein, the term “overhang” refers to a double-stranded nucleic acid molecule, which does not have blunt ends, such that the ends of the two strands are not coextensive, and such that the 5′ end of one strand extends beyond the 3′ end of the opposing complementary strand. It is possible for a linear nucleic acid molecule to have zero, one, or two, 5′ overhangs.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured, for instance, by cell viability assays, FACS analysis or DNA electrophoresis.

The terms “cancer” and “cancerous” refer to or describe the physiological condition in mammals that is typically characterized by unregulated cell growth. Examples of cancer include, without limitation, carcinoma, lymphoma, leukemia, blastoma, and sarcoma. Specific examples of such cancers include pancreatic cancer, colorectal cancer, gastrointestinal cancer, squamous cell carcinoma, small-cell lung cancer, non-small cell lung cancer, breast cancer, glioblastoma multiforme, cervical cancer, stomach cancer, bladder cancer, prostate cancer, hepatoma, and head and neck cancer. In a preferred embodiment, the cancer includes pancreatic cancer, colorectal cancer, breast cancer, ovarian cancer, prostate cancer, and lung cancer.

The term “treatment” refers to both therapeutic treatment and prophylactic or preventative measures, wherein the object is to prevent or slow down (lessen) the targeted pathologic condition or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

A “subject” is a vertebrate, preferably a mammal, more preferably a human.

The term “mammal” is used herein to refer to any animal classified as a mammal, including, without limitation, humans, higher primates, rodents, domestic and farm animals, and zoo, sports, or pet animals, such as sheep, dogs, horses, cats, cows, etc. Preferably, the mammal herein is human.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, including fragments and/or variants thereof.

The term “chemotherapeutic agent” is used herein to refer to a chemical compound useful in the treatment of cancer. Examples of chemotherapeutic agents include, without limitation, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (₁¹ and calicheamicin 2¹₁, see, e.g., Agnew Chem Intl. Ed. Engl. 33:183-186 (1994); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmo fur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “inflammatory disease” or “inflammatory disorder” refers to pathological states resulting in inflammation, typically caused by neutrophil chemotaxis. Examples of such disorders include inflammatory skin diseases including psoriasis, eczema, and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. The preferred indications include, without limitation, rheumatoid arthritis (RA), rheumatoid spondylitis, gouty arthritis and other arthritic conditions, chronic inflammation, autoimmune diabetes, multiple sclerosis (MS), asthma, systhemic lupus erythrematosus, adult respiratory distress syndrome, Behcet's disease, psoriasis, chronic pulmonary inflammatory disease, graft versus host reaction, Crohn's Disease, ulcerative colitis, inflammatory bowel disease (IBD), Alzheimer's disease, and pyresis, along with any disease or disorder that relates to inflammation and related disorders.

B. Detailed Description

In this invention, several sets of transcription factor decoys that specifically bind to a HIF transcription factor (HIF dsODNs), in particular transcription factor HIF-1 (HIF-1 dsODNs) and/or HIF-2 (HIF-2 dsODNs) were systematically developed, tested and improved.

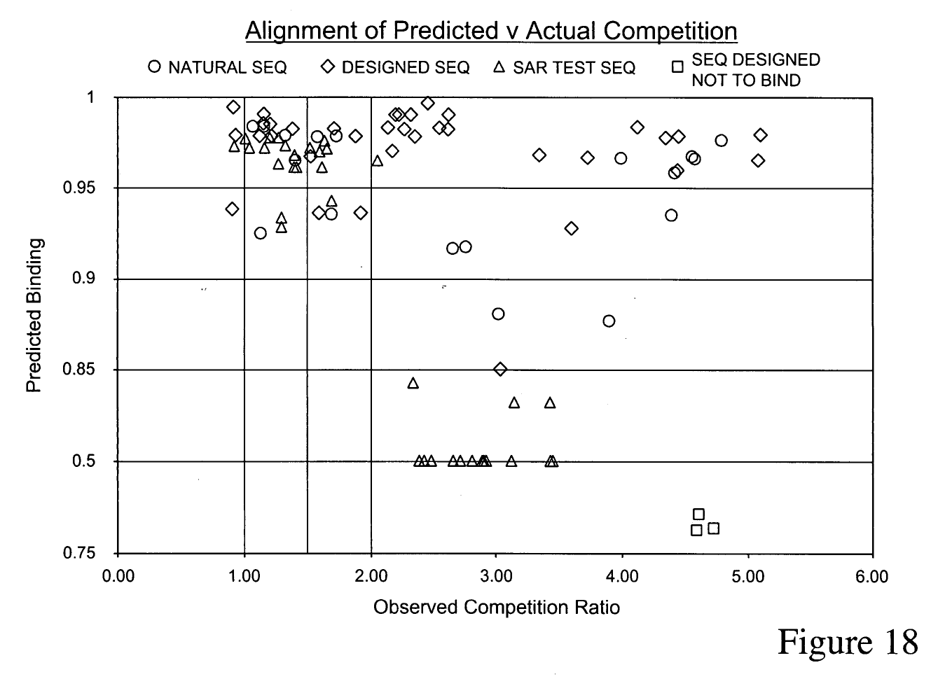

Initially, a series of HIF dsODN molecules were synthesized, with all possible bases in the core sequence (designated as positions 1-5) and the surrounding 5′ and 3′ sequences (designated as positions −1 through −4 for the 5′ and positions +1 through +3 for the 3′ sequences). Each dsODN sequence was analyzed, using bioinformatics methods which give a score of how well a decoy is predicted to bind to its HIF target. Subsequently, the ability of the HIF decoys to bind to and block the activity of a HIF transcription factor was determined in traditional binding assays (e.g. competitive binding assay), including the TransAM™ method (Active Motif, Carlsbad, Calif.), which is an ELISA-based method for detecting and quantifying transcription factor activation, as described in the Examples below, and the predicted and actual binding affinities were compared. The data, which are illustrated in FIG. 18, and discussed below.

In addition, the effect of the length and composition of the 3′ and 5′ flanks surrounding the core sequence on binding affinity was investigated.

Finally, a series of experiments were performed to assess the effect of backbone substitutions on the binding affinity of HIF decoys.

These structure-function studies lead to the identification of a series of HIF dsODN molecules with superior properties, which are promising candidates for the treatment of a variety of HIF associated diseases.

1. Desien of HIF dsODN Molecules

In one embodiment, the HIF dsODN molecules of the present invention consist of two oligonucleotide strands which are attached to each other by Watson-Crick base pairing. While typically all nucleotides in the two strands participate in the base pairing, this is not a requirement. Oligonucleotide decoy molecules, where one or more, such as 1-3 or 1 or 2 nucleotides are not involved in base pairing are also included. In addition, the double stranded decoys may contain 3′ and/or 5′ single stranded overhangs.

In another embodiment, the HIF dsODN molecules of the present invention comprise two oligonucleotide strands which are attached to each other by Watson-Crick base pairing, and are additionally covalently attached to each other at either the 3′ or the 5′ end, or both, resulting in a dumbell structure, or a circular molecule. The covalent linkage may be provided, for example, by phosphodiester linkages or other linking groups, such as, for example, phosphothioate, phosphodithioate, or phosphoamidate linkages.

Generally, the dsODN molecules of the invention comprise a core sequence that is capable of specific binding to a HIF transcription factor, such as HIF-1 and/or HIF-2, flanked by 5′ and/or 3′ sequences, wherein the core sequence consists of about 5 to 14, or about 6 to 12. or about 7 to about 10 base pairs; and the flanking sequences are about 2 to 10, or about 2 to 8, or about 6 to 10, or about 7 to 10, or about 8 to 10, or about 6 to 8, or about 7 to 8 base pairs long. The molecule typically comprises an about 12 to 28, preferably about 14 to 24 base-pair long double-stranded region composed of two fully or partially complementary strands (including the core and flaking sequences). In a particular embodiment, the 5′ flanking sequence is at least about 6 base pairs long, while the 3′ flanking sequence is at least about 6, or at least about 7 base pairs long.

Changing the core sequence (including its length, sequence, base modifications and backbone structure) it is possible to change the binding affinity of the HIF decoy molecule. In addition, changes in the flanking sequence have a genuine impact on and can significantly increase the in vivo stability of the HIF decoy molecule, and may affect binding affinity and/or specificity. In particular, the shape/structure of the HIF decoy molecule can be changed by changing the sequences flaking the core binding sequence, which can result in improved stability and/or binding affinity. The shape and structure of the DNA are influenced by the base pair sequence, length of the DNA, backbone and nature of the nucleotide (i.e. native DNA vs. modified sugars or bases). Thus, the shape and/or structure of the molecule can also be changed by other approaches, such as, for example, by changing the total length, the length of the fully complementary, double-stranded region within the molecule, by alterations within the core and flanking sequences, by changing the backbone structure and by base modifications.

The nucleotide sequences present in the decoy molecules of the present invention may comprise modified or unusual nucleotides, and may have alternative backbone chemistries. Synthetic nucleotides may be modified in a variety of ways, see, e.g. Bielinska et al. Science 250:997-1000 (1990). Thus, oxygens may be substituted with nitrogen, sulfur or carbon; phosphorus substituted with carbon; deoxyribose substituted with other sugars, or individual bases substituted with an unnatural base. Thus replacement of non-bridging oxygen atoms of the internucleotide linkage with a sulfur group (to yield a phosphorothioate linkage) has been useful in increasing the nuclease resistance of the dsODN molecule. Experiments determining the relationship between the number of sulfur modifications and stability and specificity of the HIF dsODN molecules herein are set forth in the Examples below.

In each case, any change is evaluated as to the effect of the modification on the binding ability and affinity of the oligonucleotide decoy to the HIF transcription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g. Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β, D-mannosylqueosine, 5-methoxycarbonylmethyl-2-thiouridine, 5-metoxycarbonalmethyluridine, 5-methoxyuridine, 2-methylthio-N-6-isopentenyladenosine, N-((9-beta-D-ribofuransyl-2-methylthiopurine-6-yl)carbamoyl)threonine, N-((9-beta-D-ribofuranosyl)purine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuranosylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl. Other linkages, such as phosphothioate, phosphodithioate, etc. are also possible.

The decoys of the present invention can also contain modified or analogous forms of the ribose or deoxyribose sugars generally present in polynucleotide structures. Such modifications include, without limitation, 2′-substituted sugars, such as 2′-O-methyl-, 2′-O-allyl, 2′-fluoro- and 2′azido-ribose, carboxylic sugar analogs, α-anomeric sugars, epimeric sugars, such as arabinose, xyloses, lyxoses, pyranose sugars, furanose sugars, sedoheptuloses, acyclic analogs and abasic nucleoside analogs, such as methyl riboside.

In general, the oligonucleotide decoys of the present invention are preferably comprised of greater than about 50%, more preferably greater than about 80%, most preferably greater than about 90% conventional deoxyribose nucleotides.

The HIF dsODN decoys of the present invention can be further modified to facilitate their localization, purification, or improve certain properties thereof. For example, a nuclear localization signal (NLS) can be attached to the decoy molecules, in order to improve their delivery to the cell nucleus.

In addition, the HIF decoy molecules of the invention may be conjugated with carrier molecules, such as peptides, proteins or other types of molecules, as described, for example, in the following references: Avrameas et al., J Autoimmun 16, 383-391 (2001); Avrameas et al., Bioconjug. Chem. 10: 87-93 (1999); Gallazzi et al., Bioconjug. Chem. 14, 1083-1095 (2003); Ritter, W. et al., J. Mol. Med. 81, 708-717 (2003).

The HIF decoy molecules of the invention may further be derivatized to include delivery vehicles which improve delivery, distribution, target specific cell types or facilitate transit through cellular barriers. Such delivery vehicles include, without limitation, cell penetration enhancers, liposomes, lipofectin, dendrimers, DNA intercalators, and nanoparticles.

For therapeutic applications, it is advantageous to develop specific, high affinity HIF decoy molecules that target all species of HIF.

Bioinformatics methods, using, for example, a TF binding sites matrix system, were useful as an initial tool in designing HIF dsODN molecules. However, as it will be apparent from the data provided in the Examples, such analysis was only the starting point in the design of decoy molecules that bind strongly to and are effective in inhibiting the biological activity of the target HIF transcription factor. Bioinformatics analysis had to be followed by extensive experimental structure-function studies in order to design highly effective inhibitors of HIF function.

2. Synthesis of HIF dsODN Molecules

The HIF dsODN decoy molecules of the present invention can be synthesized by standard phosphodiester or phosphoramidate chemistry, using commercially available automatic synthesizers. The specific dsODN molecules described in the example have been synthesized using an automated DNA synthesizer (Model 380B; Applied Biosystems, Inc., Foster City, Calif.). The decoys were purified by column chromatography, lyophilized, and dissolved in culture medium. Concentrations of each decoy were determined spectrophotometrically.

3. Characterization of HIF dsODN Molecules

The HIF decoy molecules of the present invention can be initially conveniently tested and characterized in a gel shift, or electrophoretic mobility shift (EMSA) assay. This assay provides a rapid and sensitive method for detecting the binding of transcription factors to DNA. The assay is based on the observation that complexes of protein and DNA migrate through a non-denaturing polyacryamide gel more slowly than free double-stranded oligonucleotides. The gel shift assay is performed by incubating a purified protein, or a complex mixture of proteins (such as nuclear extracts), with a ³²P end-labeled DNA fragment containing a transcription factor-binding site. The reaction products are then analyzed on a non-denaturing polyacrylamide gel. The specificity of the transcription factor for the binding site is established by competition experiments, using excess amounts of oligonucleotides either containing a binding site for the protein of interest or a scrambled DNA sequence. The identity of proteins contained within a complex is established by using an antibody which recognizes the protein and then looking for either reduced mobility of the DNA-protein-antibody complex or disruption of the binding of this complex to the radiolabeled oligonucleotide probe.

The ability of a HIF decoy to bind to and block the activity of a HIF transcription factor can be determined in traditional binding assays (e.g. competitive binding assay), including the TransAM™ method (Active Motif, Carlsbad, Calif.), which is an ELISA-based method for detecting and quantifying transcription factor activation. Briefly, a target sequence, in this case the HIF binding site in the EPO promoter, is immobilized on the plate, and a nuclear extract containing HIF is incubated in the wells, in the presence or absence of decoy at various concentrations calculated as the molar ratio of decoy:plate bound sequence. Positive control wells include decoy with the same sequence as the target DNA on the plate. The data obtained are presented as the ratio of the absorbance of the test decoys and the absorbance of the positive control decoy. Accordingly, lower ratios represent better binding. In this assay, typically scores up to about 1.5 are considered as indicative of very good competitive inhibitor (binding) properties, ratios around 1.2 and below being viewed as optimal. Decoy molecules, which for which the ratio of about 2 or above are generally considered poor competitive inhibitors.

The ability of a HIF decoy to block HIF activity can be further assessed in in vitro cell based assays, such as, for example, by testing its ability to reduce hypoxia-induced HIF activity in cancer cells, as described in the examples below.

In vivo efficacy can be initially tested in animal models, such as murine xenografts models using human cancer cells. This can be followed by testing in animal models of a particular target disease, followed by clinical trials to assess safety and efficacy in the treatment of the particular disease. The results of efficacy studies in various tumor models are presented in the Examples below.

4. Use of HIF dsODN Molecules

As discussed before HIF-1 has been shown to play a critical role in tumor growth, including angiogenesis and glycolysis, and metastases, and identified as a potential target for anti-cancer therapeutic strategies. (Semenza, Nature Rev. 3:721-732 (2003); Williams et al., Oncogene 21:282-90 (2002); Griffiths et al., Cancer Res. 62:688-95 (2002); Welsh et al., Mol. Cancer Ther. 2:235-43 (2003)). HIF-1 has been shown to be overexpressed in breast cancer and potentially associated with more aggressive tumors (Bos et al., J. Natl. Cancer Inst. 93:309-314 (2001)). In addition, HIF-1 has been recently identified as a critical link between inflammation and oncogenesis (Jung et al., The FASEB Journal Express Article 10.1096/fj.03-0329fjc, published online Sep. 4, 2003). HIF-1α overexpression in biopsies of brain, breast, cervical, esophageal, oropharyngeal and ovarian cancers is correlated with treatment failure and mortality. Increased HIF-1 activity promotes tumor progression, and inhibition of HIF, such as HIF-1 and/or HIF-2 could represent a novel approach to cancer therapy. Therefore, blocking HIF-1 and/or HIF-2 by the decoy molecules of the present invention finds utility in the prevention and treatment of cancer, offering a new anti-cancer strategy, either alone or in combination with other treatment options. Inhibition of HIF-1 and/or HIF-2 by administering the dsODN molecules of the present invention may also enhance the efficacy of other cancer therapies, such as radiation therapy and/or treatment with chemotherapeutic agents. Specific cancer targets include, without limitation, solid tumor malignancies and Non-Hodgkin's lymphoma.

As shown in the examples below, the HIF dsODN molecules of the present invention effectively inhibit tumor growth in various cell-based assays and xenograft models, and are thus promising anti-cancer agents for the treatment of a variety of tumors, including, without limitation, pancreatic, colon, and lung cancer.

In addition, HIF-1 has been identified as a target for diseases in general in which hypoxia is a major aspect, such as, for example, heart disease and stroke (Giaccia et al., Nat. Rav. Drug Discov. 2:803-822 (2003)), and chronic lung disease. Accordingly, the HIF decoy molecules of the present invention can also be used for the prevention and treatment of hypoxia-associated diseases and pathologic conditions, such as, for example, cardiovascular diseases (including ischemic cardiovascular diseases), such as myocardial ischemia, myocardial infarction, congestive heart failure, cardiomyopathy, cardiac hypertrophy, and stroke.

HIF decoy molecules additional find utility in ophthalmology, including diabetic retinopathy, which is the leading cause of blindness in the United States. Additional opthalmologic targets include Age-related Macular Degeneration (AMD), and corneal neovascularization associated with transplants.

HIF dsODN molecules find additional use in the prevention and treatment of pathogenic blood vessel growth, associated, for example, with psoriasis, corneal neovascularization, infection or trauma.

Increased angiogenesis is also a key component of synovitis and bone modeling in arthritis. Preclinical studies of angiogenesis inhibitors in animals models of inflammatory arthritis support the hypothesis that inhibition of neovascularization may reduce inflammation and joint damage. Therefore, additional therapeutic targets include inflammatory diseases, including arthritis, such as rheumatoid arthritis (RA), and musculoskeletal disorders. For further details see, e.g. Walsh and Haywood, Curr Opin Investig Drugs. 2(8): 1054-63 (2001). In addition, similar to tumor growth, endometriotic implants require neovascularization to establish, grow and invade. This process can be blocked by the HIF decoys of the present invention. See also, Taylor et al. Ann NY Acad Sci. 955:89-100 (2002).

For further details of HIF, sch as HIF-1, associated diseases see, e.g. Semenza, G. K., J Appl Physiol 88:1474-1480 (2000).

5. Delivery of the HIF dsODN Molecules

A possible mode of delivering the HIF decoys of the present invention is pressure-mediated transfection, as described, for example, in U.S. Pat. Nos. 5,922,687 and 6,395,550, the entire disclosures of which are hereby expressly incorporated by reference. In brief, the HIF decoy molecules are delivered to cells in a tissue by placing the decoy nucleic acid in an extracellular environment of the cells, and establishing an incubation pressure around the cells and the extracellular environment. The establishment of the incubation pressure facilitates the uptake of the nucleic acid by the cells, and enhances localization to the cell nuclei.

More specifically, a sealed enclosure containing the tissue and the extracellular environment is defined, and the incubation pressure is established within the sealed enclosure. In a preferred embodiment, the boundary of the enclosure is defined substantially by an enclosing means, so that target tissue (tissue comprising the target cell) is subjected to isotropic pressure, and does not distend or experience trauma. In another embodiment, part of the enclosure boundary is defined by a tissue. A protective means such as an inelastic sheath is then placed around the tissue to prevent distension and trauma in the tissue. While the incubation pressure depends on the application, incubation pressures about 300 mmHg-1500 mmHg above atmospheric pressure, or at least about 100 mmHg above atmospheric pressure are generally suitable for many applications.

The incubation period necessary for achieving maximal transfection efficiency depends on parameters such as the incubation pressure and the target tissue type. For some tissue, such as human vein tissue, an incubation period on the order of minutes (>10 minute) at low pressure (about 0.5 atm) is sufficient for achieving a transfection efficiency of 80-90%. For other tissue, such as rat aorta tissue, an incubation period on the order of hours (>1 hour) at high pressure (about 2 atm) is necessary for achieving a transfection efficiency of 80-90%.

Suitable mammalian target tissue for this type of delivery includes blood vessel tissue (in particular veins used as grafts in arteries), heart, bone marrow, and normal and tumor connective tissue, liver, genital-urinary system, bones, muscles, gastrointestinal organs, endocrine and exocrine organs, synovial tissue and skin. A method of the present invention can be applied to parts of an organ, to a whole organ, or to a whole organism. In one embodiment a nucleic acid solution can be perfused into a target region (e.g. a kidney) of a patient, and the patient is subject to pressure in a pressurization chamber.

For other applications, the HIF decoys of the present invention can be administered by other conventional techniques. For example, retroviral transfection, transfection in the form of liposomes are among the known methods suitable for transfection. For details see also Dzau et al., Trends in Biotech 11:205-210 (1993); or Morishita et al., Proc. Natl. Acad. Sci. USA 90:8474-8478 (1993). When administered in liposomes, the decoy concentration in the lumen will generally be in the range of about 0.1 μM to about 50 μM per decoy, more usually about 1 μM to about 10 μM, most usually about 3 μM.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides. In general, dosage is from 0.01 μg to 100 g per kg of body weight. Persons of ordinary skill in the art can easily estimate repetition rates for dosing based on measured residence times and concentrations of the drug in bodily fluids or tissues. In addition to the potency of the specific decoy molecule delivered, the effective dose will depend on the target disease, the route of delivery, the formulation used, the severity of the disease, the age, sex, and overall condition of the patient to be treated.

The decoys may be administered as compositions comprising individual decoys or mixtures of decoys. Usually, a mixture contains up to 6, more usually up to 4, more usually up to 2 decoy molecules.

In cancer therapy, the administration of the HIF decoy molecules can be combined with other treatment options, including surgery, treatment with chemotherapeutic anticancer agents and/or radiation therapy.

Cancer treatment with HIF decoys may specifically include combination therapy with anti-angiogenic agents (angiogenesis inhibitor), such as, for example, anti-EGF and anti-VEGF agents, matrix metalloproteinase inhibitors, vascular targeting agents, integrin antagonists, and the like. Solid tumors are known to contain areas of viable and necrotic tissue. Blocking blood supply to the tumor by anti-angiogenic agents results in severe hypoxia throughout the cancer tissue. Since hypoxia is known to induce HIF, inhibition of angiogenesis, by increasing hypoxia, increases the therapeutic window for HIF decoy treatment.

Angiogenesis inhibitors that are commercially available or are under development include, for example, Avastin™ (bevacizumab, Genentech, Inc.), an anti-VEGF monoclonal antibody; angiostatin; endostatin; Panzem® (2-methoxyestradiol, EntreMed, Inc.); Iressa® (gefitinib, AstraZeneca), and thalidomide. Combination therapies might result in reduction of the effective dose, which in turn might reduce toxic side-effects or other complications.

Further details of the invention will be apparent from the following non-limiting Examples.

### EXAMPLE 1

Design and Testing of HIF Decoy Molecules

Design

Initially, the HIF-1 binding DNA consensus sequences were selected from publications of HIF-1 related DNA-protein interactions, and were chosen from the published sequence set summarized in BioBase TRANSFAC (versions 7.2 and 8.2) database. Their corresponding regulatory region localizations have been confirmed and the extended flanking genomic DNA sequences retrieved from the most updated genome database (see Table 1) (for human, version July 2003; for mouse, version February, 2003; for rat, version June, 2003).

TABLE 1
Identified HIF-1 binding sites and corresponding flanking sequences.

| | | |
|---|---|---|
| Genes/ | | SEQ ID |
| Designations | Sequences | NO: |

| | | |
|---|---|---|
| ADM_825 | GTGTGCTCCCAGTCAGTCAATCCTCACGTTTATGATGGATGAATGAAGGCAG | 2 |
| EDN1 | TTGTGTTATTAGTCACCAACAGGCAACGTGCAGCCGGAGATAAGGCCAG | 3 |
| HMOX1_2 | ATCCCCCCGCCCACAGAGAGGACGTGCCACGCCAGCACGTCCGCTCTCCTTGCCAG | 4 |
| ADM_1 | TGTGCTCCCAGTCAGTCAATCCTCACGTTTATGATGGATGAATGAAGGCAGTCAGGT | 5 |
| ADM_1203 | GTGATGAAAGAGCACAAACGGGTGACAAACGTGTCTAGCGTGATTCATCATGAACAGGCACA | 6 |
| ADM_863 | TGCTTGGTAAACTGTAAAATGATTAGCATACGTGAAGCGTTAGTGTGCTCCCTGGCA | 7 |
| Adra1b | GAGCGAGCCGCTGGGTGCAGGCAGGCGACGTGCTGCCGGGCTAGGCTGCCCGGGGGAGATGA | 8 |
| ALDA_1 | GTGGTCCGAGTCACGTCCGAGGGGG | 9 |
| ALDA_2 | CTTCACGTGCGGGGACCAGGGACCGT | 10 |
| ENO1_1 | CGCAGGCGCAGGCGGCGCACGTGGCC | 11 |
| ENO1_364 | GAGTGCGTGCGGGACTCGGAGTACGTGACGGAGCCCCGAGCTCTCATGCC | 12 |
| ENO1_383 | GGGGCCCCAGAGCGACGCTGAGTGCGTGCGGGACTCGGAGTACGTGACGGAGC | 13 |
| ENO1_409 | GCAAGGTCGAGGGCCGGACGTGGGGCCCCAGAGCGACGCTGAGTGCGTGCGG | 14 |
| EPO_2 | GGGGCGTGAGCGGGGCTGCTGCAGACGTGCGTGTGGGTCATGGGGGCTGCTC | 15 |
| EPO_1 | GCCCTACGTGCTGTCTCACACAGCCTGTCTGACCTCTCGACCT | 16 |
| ET_1 | CTCCGGCTGCACGTTGCCTG | 17 |
| FLT1_1 | ATGGAGACATAATTGAGGAACAACGTGGAATTAGTGTCATAGCAAATGATCTAGG | 18 |
| Hmox1 | GAGCGGACGTGCTGGCGTGGCACGTCCTCTC | 19 |
| LDH1(A)_1 | GACGCCCGCCCCCGGCCCAGCCTACACGTGGGTTCCCGCACGTCCGCTGGG | 20 |
| LDH1(A)_2 | CGTCAGAGTGGGAGCCCAGCGGACGTGCGGGAACCCACGTGTAGGCTGGGC | 21 |
| Nos2 | GTGACTACGTGCTGCCTAGGGGCCACTGCC | 22 |
| PAl-1 | CCTGAATGCTCTTACACACGTACACACACAGAGCAGC | 23 |
| PFKL | CCGGGTAGCTGGCGTACGTGCTGCAG | 24 |
| PGK1_1 | CCTTGCGGTTCGCGGCGTGCCGGACGTGACAAACGGAAGCCGCACGTCTCACTA | 25 |
| PGK1_2 | CCTTGCGGTTCGCGGCGTGCCGGACGTGACAAACGGAAGCCGCACGTCTCACTA | 26 |
| PPARA | CTGCCAGTGCACGTCAGTGG | 27 |
| RTP801 | GCCCGGCCGCTGTCACCGGGCAGGAGAGAACGTTGCTTACGTGCGCCCGGAGTCCATTGGCCAAGGCGGGCC | 28 |
| Slc2a1_p1 | AAGGCCCTGGGTCCACAGGCGTGCCGTCTGACACGCATCAGGCAGGCACTC | 29 |
| Slc2a1_rat | CCATTTCTAGGGCCTTGGGTCCACAGGCGTGCTGGCTGACACGCATCAGGCCG | 30 |
| TF_1 | TTCCTGCACGTACACACAAAGCGCACGTATTTC | 31 |
| TFREC | TCAGAGCACCTCGCGAGCGTACGTGCCTCAGGAAGTGACGCACAGCCCCCCTGGGGGCCGG | 32 |
| VEGF | GGGTTTTGCCAGACTCCACAGTGCATACGTGGGCTCCAACAGGTCCTCTTCCCTCCCAGTCACTG | 33 |
| Ahrr | GTCTGCCGCTTCGGGCGGGGCGGCTCGCGTGCTGGGGTGGGGCTTTCCTTCTCTAGTCT | 136 |
| Ahrr | AGATCAGAATAGGTAACCAAGACTTAGCGTGTTCCTCCTTTCTTTGCGCTATAGAGTTGT | 137 |
| Ahrr | CTGTCTCCTGAGTGTTGGGATTAAAGGCGTGTGCCGTCACCACCTGGCTCACTACCACA | 138 |
| Aldh3a1 | CCTCAGAGTCCTTCCCCAGGAAGTTCGCGTGACGAAGATAAAACAATATTCAAATGTGG | 139 |
| Aldh3a1 (Aldh3) | TGCACTAATGCACACCCACCACTAATGCaTGCCCCATCACTAATGCACACCCCCATCAC | 140 |
| CTSD (CATH-D) | CCGGACCGGTCACGTGGGCGCGGCCGGCGTGCGCGGGGCGGGGCGGAGCGGGGCCTGGC | 141 |
| Cyp1a1 | CACCCACGGCTCCCCTCCCCCAGCTAGCGTGACAGCACTGGGACCCGCGCCCGGTAGTG | 142 |
| Cyp1a1 | GGCTGGGGACAAGGTGCCCCGGAGTTGCGTGAGAAGAGCCTGGAGGCCTGCGCAGCCAC | 143 |
| Cyp1a1 | GGCTGGGGACAAGGTGCCCCGGAGTTGCGTGAGAAGAGCCTGGAGGCCCGCGCAGCCAC | 144 |
| Cyp1a1 | GGCACGCACACAGGTTCCTGAGGCTAGCGTGCGTAAGCCTGCTCCATCCTCTGGGGGCA | 145 |
| Cyp1a1 | AGCCCCAGACCCCCTCCTGCTGTCTCGCGTGGATCCTTCCTCCACCCTTTCCTCCACCA | 146 |
| Cyp1a1 | TTCCCTGGATTACTGAGTCCAAGCTCGCGTGAGAAGCGCAACGACCCCAGCCCAGAGGT | 147 |
| CYP1A1 | CATTTTTGCACCCACTGGAACGCTGGGCGTGCAGATGCCTCCCCAGCGCTACAGCCTAC | 148 |
| CYP1A1 | TTCCCGGGGTTACTGAGTCCCGGCTCGCGTGAGAAGCGCTGCGACCCCAGCCCTGAGGT | 149 |
| CYP1A1 | GCCCCCACCCTACCCCCGGCTAGCTTGCGTGCGCCGGCGACATCCCTCTAGGGGGCAGA | 150 |
| CYP1A1 | GCCCGGAGGCGCGGTGCCCAGGCGTTGCGTGAGAAGGACCGGAGGCCCGCGCAGCCACC | 151 |
| CYP1A1 | GCCCCGCCCCGACTCCCTCCCCCCTCGCGTGACTGCGAGCCCCCGCGCCGGGCCGGGGA | 152 |
| FOS | TACGCAGCAGGGCAGGAGATTGGGGGGCGTGGCACACTCTGGAGCACCTTGCCTCCCCA | 153 |
| IL-2 | GTATGTGTGTGTGAGCATGTTTATGTGCGTGTGTATATGAGTGTGTGTGTGTGCATGTA | 154 |
| Ldh1 | GGAGCCCAGCGGACGTGCGGGAACCCACGTGTAGGCTGGGCCGGGGGCGGGCGTCCAGC | 155 |
| Polk | TCCAGCCTGGCTTCCGATTCTGCCTTGCGTGTTTGTGACGAGCCAGCGAGCCGGGACGT | 156 |
| Polk | AGGCAAGTGGGCTTCTTTTGAGAGTTGCGTGCCCCTTTCCGTCCAGCCTGGCTTCCGAT | 157 |
| Ugt1a1 (Ugt1) | GTCTTTGATCAAGATCCTTTTCTGCTGCGTGTGGTTAAAACATACAACAAAGTCAAAAG | 158 |
| UGT1A6 | AAACTTGTGGGTGGGAACAGGAACTCGCGTGcCAGCCAGGTGTGCATGACTAGCTCTGGG | 159 |
| Vegf | GGGCTCTGCCAGACTCCACAGTGCATACGTGGGCTTCCACAGGTCGTCTCCCTCCGGGC | 160 |

Construction of Matrix of Core Consensus Binding Sites

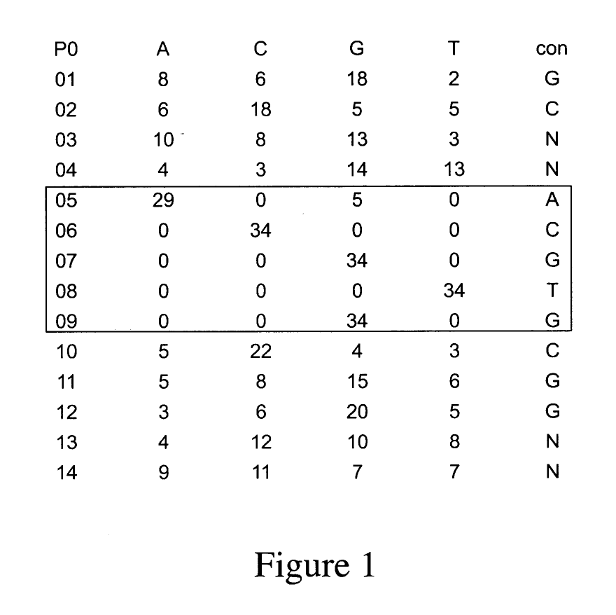

To define the base-composition near the core binding sites of HIF, the core binding sites based on the above available HIF-1 core binding sequences were computationally aligned. Based on the alignment, a table or “matrix” was created that computationally describes the base composition for both the core and the immediate-flanking regions (see FIG. 1). The analysis was conducted using the most updated version (8.2, June 2004) of the TRANSFAC database (see, e.g. Heinemeyer et al., Nucleic Acids Res. 27:318-22 (1999); Knuppel et al., J. Comput. Biol. 1:191-8 (1994); Matys et al., Nucleic Acids Res. 31:374-8 (2003); Schacherer et al., Bioinformatics 17:1053-7 (2001)). TRANSFAC collects position-weight-matrices for DNA-TF binding. The tool Match (Kel et al., Nucleic Acids Res. 31:3576-3579 (2003) uses these matrices to computationally predict the binding affinity). FIG. 1 statistically suggests the probability that a given base will be found at a given position.

Analysis of Crystal Structure of HIF-1 binding motif

HIF-1 is in a family of basic helix-loop-helix (bHLH) DNA binding proteins. The amino acids located from position 30 to position 70 (out of total 826 for the HIF-1a subunit) are responsible for the DNA recognition and binding affinity. While there is no crystal structure of the DNA binding motif for HIF-1, the available structural information of other bHLH members that share a similar DNA binding motifs provides useful structural information (Michel et al., Theor Chem Acc. 101:51-56 (1999); Michel et al., J. Biomolecular Structure & Dynamics 18:169-179 (2000); Michel et al., Biochimica et Biophysica Acta 1578:73-83 (2002)). These studies suggested the importance of several residues located in the binding motif of HIF-1. The known core binding sequence is CGTG (SEQ ID NO: 134), however it has been found that the central core ACGTG (SEQ ID NO: 126) is essential for maximum binding of the HIF-1 complex (HIF-1α and ARNT), and the base-composition immediately 5-prime upstream from the core is also very important for the specificity and affinity of HIF-1 binding. DNA-footprint studies also suggested that the 5-prime upstream region could be important for HIF-1 induced gene expression. Therefore, candidate decoys with varying sequences and lengths of the 5′ flank were designed and tested.

Sequences of Initial HIF-1a Decoys

Based on the knowledge from published HIF-1 binding studies, from available HIF-1 core binding sequences, from the computational core binding matrix, from the model of crystal structures about bHLH family, and from specific bioinformatics approaches (to exclude the decoy that may binding to other transcription factors), a set of decoys were generated for initial screening (see Table 2A). These decoys include a “mutation decoy”, “scramble decoys”, decoys with different length at their 5′ or 3′ end, and decoys with alternative base composition at or flank the core region.

TABLE 2A
Initial sequences for screening

| | | |
|---|---|---|
| | | SEQ |
| # | Sequences | ID |

| | | |
|---|---|---|
| 801 | GCC CTA CGT GCT GTC TGA | 34 |
| 802 | TGA GAG AGC ACG TAG GGC | 35 |
| 803 | CTG TCC TCC GAC TGC ATG | 36 |
| 804 | CAT GCA GTC GGA GGA GAG | 37 |
| 805 | CCC CCT CGG ACG TGA CTG GGA CCA C | 38 |
| 806 | G TGG TCC GAG TGA CGT CCG AGG GGG | 39 |
| 807 | TCT GTA CGT GAG CAC ACT CAC CTC | 40 |
| 808 | GAG GTG AGT GTG GTC ACG TAG AGA | 41 |
| 809 | AGG GCC GGA CGT GGG GCC CC | 42 |
| 810 | GG GGC CCC ACG TCC GGC CCT | 43 |
| 811 | ACG CTG AGT GCG TGG GGG AG | 44 |
| 812 | GT CCC GCA CGC AGT CAG CGT | 45 |
| 813 | GCC CTA CGT GCT GTC TCA CAC AGC | 46 |
| 814 | GCT GTG TGA GAC AGC ACG TAG GGC | 47 |
| 815 | GTG AGA CGT GCG GCT TCC GTT TG | 48 |
| 816 | CA AAC GGA AGC CGC ACG TCT CAC | 49 |
| 817 | CTG CCG ACG TGC GCT CCG GAG | 50 |
| 818 | CTC CGG AGC GCA CGT CGG CAG | 51 |
| 819 | GAA ATA CGT GCG CTT TGT GTG TAC GTG CAG GAA | 52 |
| 820 | TTC CTG CAC GTA CAC ACA AAG CGC ACG TAT TTC | 53 |
| 821 | CGC GAG CGT ACG TGC CTC AGG | 54 |
| 822 | CCT GAG GCA CGT ACG CTC GCG | 55 |
| 823 | TGC ATA CGT GGG CTC CAA CAG | 56 |
| 824 | CTG TTG GAG CCC ACG TAT GCA | 57 |
| 825 | AGG AGA CGT GCG AGA A | 58 |
| 826 | T TCT CGC ACG TCT CCT | 59 |
| 827 | AGG TTA CGT GCG GAC A | 60 |
| 828 | T GTC CGC ACG TAA CCT | 61 |
| 829 | AGG AGA CGT GCT GCC T | 62 |
| 830 | A GGC AGC ACG TCT CCT | 63 |
| 831 | TCC AAT ACG TGC AGT ACT | 64 |
| 832 | AGT ACT GCA CGT ATT GGA | 65 |
| 833 | TCC AAT GCG TGC AGT ACT | 66 |
| 834 | AGT ACT GCA CGC ATT GGA | 67 |
| 835 | GGC CAG ACG TGC CAC CGG | 68 |
| 836 | CCG GTG GCA CGT CTG GCC | 69 |
| 837 | AGG CAA CGT GCA GCC G | 70 |
| 838 | C GGC TGC ACG TTG CCT | 71 |
| 839 | AGG CAA TAC GCA GCC G | 72 |
| 840 | C GGC TGC GTA TTG CCT | 73 |
| 841 | AGC GGA CGT GCA GAA GTT GCA CGT CCT CT | 74 |
| 842 | AG AGG ACG TGC AAC TTC TGC ACG TCC GCT | 75 |
| 843 | GTG CAT ACG TGG GCT CCA | 76 |
| 844 | TGG AGC CCA CGT ATG CAC | 77 |
| 845 | GAG CGT ACG TGC CTC AGG | 78 |
| 846 | CCT GAG GCA CGT ACG CTC | 79 |
| 847 | GGA ACA ACG TGG AAT TAG | 80 |
| 848 | CTA ATT CCA CGT TGT TCC | 81 |
| 849 | GCC TAC ACG TGG GTT CCC | 82 |
| 850 | GGG AAC CCA CGT GTA GGC | 83 |
| 851 | CGG AGT ACG TGA CGG AGC | 84 |
| 852 | GCT CCG TCA CGT ACT CCG | 85 |
| 853 | TTG CTT ACG TGC GCC CGG | 86 |
| 854 | CCG GGC GCA CGT AAG CAA | 87 |
| 855 | GTG TGT ACG TGC AGG AAA | 88 |
| 856 | TTT CCT GCA CGT ACA CAC | 89 |
| 857 | GCG GAC GTG CGG GAA CCC ACG TGT AGG | 90 |
| 858 | CCT ACA CGT GGG TTC CCG CAC GTC CGC | 91 |
| 859 | ACC GTA CGT GCT GAT C | 92 |
| 860 | G ATC AGC ACG TAC GGT | 93 |
| 861 | CTA ATA CGT GCC GCT G | 94 |
| 862 | C AGC GGC ACG TAT TAG | 95 |
| 863 | AGC AGA CGT GCA GGA T | 96 |
| 864 | A TCC TGC ACG TCT GCT | 97 |
| 865 | AGC AGA CGT GCA GGC A | 98 |
| 866 | T GCC TGC ACG TCT GCT | 99 |
| 867 | TCC GTA CGT GCT GCA C | 100 |
| 868 | G TGC AGC ACG TAC GGA | 101 |
| 869 | AGC AGA CGT GCA GGG T | 102 |
| 870 | A CCC TGC ACG TCT GCT | 103 |
| 871 | ACC GTA CGT GCT GCC A | 104 |
| 872 | T GGC AGC ACG TAC GGT | 105 |
| 873 | TCC GTA CGT GCT GCG T | 106 |
| 874 | A CGC AGC ACG TAC GGA | 107 |
| 875 | TGC AGA CGT GCA GGT C | 108 |
| 876 | G ACC TGC ACG TCT GCA | 109 |
| 877 | ACC GTA CGT GCT GCT A | 110 |
| 878 | T AGC AGC ACG TAC GGT | 111 |
| 879 | GGC TGC TGC AGA CGT GCA GGT C | 112 |
| 880 | G ACC TGC ACG TCT GCA GCA GCC | 113 |
| 881 | GGC TGC AGG AGA CGT GGA GAA | 114 |
| 882 | TTC TCC ACG TCT CCT GCA GCC | 115 |
| 883 | AGA AGA CGT GCA GGA T | 116 |
| 884 | A TCC TGC ACG TCT TCT | 117 |
| 885 | TAC AGA CGT GCA GGT C | 118 |
| 886 | G ACC TGC ACG TCT GTA | 119 |
| 887 | GGC TGC ACC GTA CGT GCT GAT C | 120 |
| 888 | G ATC AGC ACG TAG GGT GCA GCC | 121 |
| 889 | TGC ATA CGT GCA GGT C | 122 |
| 890 | G ACC TGC ACG TAT GCA | 123 |
| 891 | GGC TGC TGC ATA CGT GCA GGT C | 124 |
| 892 | G ACC TGC ACG TAT GCA GCA GCC | 125 |
| 893 | CACGA GCGTACGTGC CTCAGG | 161 |
| 895 | CACCA GCGTACGTGC CTCAGG | 162 |
| 896 | CCT GAG GCA CGT ACG CTG GTG | 135 |
| 897 | CCA GCGTACGTGC CTCAGG | 163 |
| 899 | CGA GCGTACGTGC CTCAGG | 164 |
| 901 | CACGT GCATACGTGG GCTCCA | 165 |
| 903 | CACCT GCATACGTGG GCTCCA | 166 |
| 905 | GATCG CCCTACGTGC TGTCTCAGAT C | 167 |
| 907 | GAAAT ACGTGCGTGT GTACGTGCAG G | 168 |
| 909 | AGCGG ACGTGCAGAT GCACGTCCTC T | 169 |
| 911 | CACA GCGTACGTGC TGTCTCA | 170 |
| 913 | CAGGCTCC GACTACGGCT GAC | 171 |
| 915 | AGA TCCGACGTAC CGACCAAG | 172 |
| 917 | CTAAGCG AGTAGCGAGT AGCC | 173 |
| 919 | CGCT ACGAGCTCTA CTCCAGG | 174 |
| 921 | CGCTCG ACGAGCTCTA CTCCA | 175 |
| 923 | CACCA GCGTAAAAGC CTCAGG | 176 |
| 925 | CCA GCGTACGTGC CTCAGG | 177 |
| 927 | CCA GCGTTCGTGC CTCAGG | 178 |
| 929 | CCA GCGTGCGTGC CTCAGG | 179 |
| 931 | CCA GCGTCCGTGC CTCAGG | 180 |
| 933 | CCA GCGTATGTGC CTCAGG | 181 |
| 935 | CCA GCGTAAGTGC CTCAGG | 182 |
| 937 | CCA GCGTAGGTGC CTCAGG | 183 |
| 939 | CCA GCGTACATGC CTCAGG | 184 |
| 941 | CCA GCGTACCTGC CTCAGG | 185 |
| 943 | CCA GCGTACTTGC CTCAGG | 186 |
| 945 | CCA GCGTACGAGC CTCAGG | 187 |
| 947 | CCA GCGTACGCGC CTCAGG | 188 |
| 949 | CCA GCGTACGGGC CTCAGG | 189 |
| 951 | CCA GCGTACGTCC CTCAGG | 190 |
| 953 | CCA GCGTACGTAC CTCAGG | 191 |
| 955 | CCA GCGTACGTTC CTCAGG | 192 |
| 957 | CCA GCGCACGTGC CTCAGG | 193 |
| 959 | CCA GCGAACGTGC CTCAGG | 194 |
| 961 | CCA GCGGACGTGC CTCAGG | 195 |
| 963 | CCA GCCTACGTGC CTCAGG | 196 |
| 965 | CCA GCTTACGTGC CTCAGG | 197 |
| 967 | CCA GCATACGTGC CTCAGG | 198 |
| 969 | CCA GGGTACGTGC CTCAGG | 199 |
| 971 | CCA GTGTACGTGC CTCAGG | 200 |
| 973 | CCA GAGTACGTGC CTCAGG | 201 |
| 975 | CCA CCGTACGTGC CTCAGG | 202 |
| 977 | CCA ACGTACGTGC CTCAGG | 203 |
| 979 | CCA TCGTACGTGC CTCAGG | 204 |
| 981 | CCA GCGTACGTGG CTCAGG | 205 |
| 983 | CCA GCGTACGTGT CTCAGG | 206 |
| 985 | CCA GCGTACGTGA CTCAGG | 207 |
| 987 | CCA GCGTACGTGC CTCAGG | 208 |
| 989 | CCA GCGTACGTGC CTCAGG | 209 |
| 991 | CCA GCGTACGTGC ATCAGG | 210 |
| 993 | CCA GCGTACGTGC CACAGG | 211 |
| 995 | CCA GCGTACGTGC CGCAGG | 212 |
| 997 | CCG GGGCACGTGC CCCAGG | 213 |
| 999 | CCA TGGCACGTGC CTCAGG | 214 |

The sequences listed next to each other in the foregoing Table 2A (e.g. 801/802; 803/804, etc.) are complementary, and form the two strands of one double-stranded oligonucleotide decoy.

Table 2B is a different presentation of the decoy sequences prepared, showing the core sequences lined up for better understanding:

TABLE 2B
Augment of decoy sequences

| | | | | |
|---|---|---|---|---|
| decoy # | transAM ratio(2.5 pM) | Matrix | aligned sequence | seq ID |

| | | | | |
|---|---|---|---|---|
| 801 | 4 | 0.97 | G CCCTACGTGC TGTCTCA | 34 |
| 813 | 4.6 | 0.97 | G CCCTACGTGC TGTCTCACAC AGC | 46 |
| 905 | 1.54 | 0.97 | GATCG CCCTACGTGC TGTCTCAGAT C | 167 |
| 911 | 1.16 | 0.99 | CACA GCGTACGTGC TGTCTCA | 170 |
| 807 | 2.76 | 0.92 | T CTGTACGTGA CCACACTCAC CTC | 40 |
| 835 | 2.91 | 0.94 | GG CCAGACGTGC CACCGG | 68 |
| 915 | 4.62 | 0.77 | AGA TCCGACGTAC CGACCAAG | 172 |
| 859 | 2.37 | 0.98 | A CCGTACGTGC TGATC | 92 |
| 867 | 2.63 | 0.99 | T CCGTACGTGC TGCAC | 100 |
| 873 | 2.33 | 0.99 | T CCGTACGTGC TGCGT | 106 |
| 871 | 2.21 | 0.99 | A CCGTACGTGC TGCCA | 104 |
| 877 | 2.23 | 0.99 | A CCGTACGTGC TGCTA | 110 |
| 805 | 1.13 | 0.93 | CCCCC TCGGACGTGA CTCGGACCAC | 38 |
| 821 | 1.33 | 0.98 | CGCGA GCGTACGTGC CTCAGG | 54 |
| 893 | 0.94 | 0.98 | CACGA GCGTACGTGC CTCAGG | 161 |
| 895 | 1.13 | 0.98 | CACCA GCGTACGTGC CTCAGG | 162 |
| 899 | 1.34 | 0.98 | CGA GCGTACGTGC CTCAGG | 164 |
| 923 | 4.2 | 0.59 | CACCA GCGTAAAAGC CTCAGG | 176 |
| 897 | 1.23 | 0.98 | CCA GCGTACGTGC CTCAGG | 163 |
| 925 | 1.28 | 0.98 | CCA GCGTACGTGC CTCAGG | 177 |
| 927 | 3.16 | 0.83 | CCA GCGTTCGTGC CTCAGG | 178 |
| 931 | 3.44 | 0.83 | CCA GCGTCCGTGC CTCAGG | 180 |
| 929 | 2.35 | 0.84 | CCA GCGTGCGTGC CTCAGG | 179 |
| 993 | 0.93 | 0.97 | CCA GCGTACGTGC CACAGG | 211 |
| 995 | 0.92 | 0.99 | CCA GCGTACGTGC CGCAGG | 212 |
| 957 | 1.41 | 0.97 | CCA GCGCACGTGC CTCAGG | 193 |
| 959 | 2.07 | 0.97 | CCA GCGAACGTGC CTCAGG | 194 |
| 961 | 1.65 | 0.98 | CCA GCGGACGTGC CTCAGG | 195 |
| 945 | 3.14 | 0.8 | CCA GCGTACGAGC CTCAGG | 187 |
| 947 | 2.94 | 0.8 | CCA GCGTACGCGC CTCAGG | 188 |
| 949 | 2.73 | 0.8 | CCA GCGTACGGGC CTCAGG | 189 |
| 939 | 2.93 | 0.8 | CCA GCGTACATGC CTCAGG | 184 |
| 941 | 3.45 | 0.8 | CCA GCGTACCTGC CTCAGG | 185 |
| 943 | 2.83 | 0.8 | CCA GCGTACTTGC CTCAGG | 186 |
| 951 | 2.91 | 0.8 | CCA GCGTACGTCC CTCAGG | 190 |
| 953 | 2.67 | 0.8 | CCA GCGTACGTAC CTCAGG | 191 |
| 955 | 2.4 | 0.8 | CCA GCGTACGTTC CTCAGG | 192 |
| 975 | 1.63 | 0.96 | CCA CCGTACGTGC CTCAGG | 202 |
| 977 | 1.43 | 0.96 | CCA ACGTACGTGC CTCAGG | 203 |
| 979 | 1.41 | 0.96 | CCA TCGTACGTGC CTCAGG | 204 |
| 963 | 1.17 | 0.97 | CCA GCCTACGTGC CTCAGG | 196 |
| 965 | 1.61 | 0.97 | CCA GCTTACGTGC CTCAGG | 197 |
| 967 | 1.53 | 0.97 | CCA GCATACGTGC CTCAGG | 198 |
| 987 | 1.16 | 0.99 | CCA GCGTACGTGC GTCAGG | 208 |
| 989 | 1.21 | 0.98 | CCA GCGTACGTGC TTCAGG | 209 |
| 991 | 1.02 | 0.98 | CCA GCGTACGTGC ATCAGG | 210 |
| 969 | 1.67 | 0.97 | CCA GGGTACGTGC CTCAGG | 199 |
| 971 | 1.05 | 0.97 | CCA GTGTACGTGC CTCAGG | 200 |
| 973 | 1.34 | 0.97 | CCA GAGTACGTGC CTCAGG | 201 |
| 981 | 1.7 | 0.94 | CCA GCGTACGTGG CTCAGG | 205 |
| 985 | 0.91 | 0.94 | CCA GCGTACGTGA CTCAGG | 207 |
| 983 | 1.3 | 0.93 | CCA GCGTACGTGT CTCAGG | 206 |
| 933 | 3.47 | 0.8 | CCA GCGTATGTGC CTCAGG | 181 |
| 935 | 2.43 | 0.8 | CCA GCGTAAGTGC CTCAGG | 182 |
| 937 | 2.49 | 0.8 | CCA GCGTAGGTGC CTCAGG | 183 |
| 845 | 1.59 | 0.98 | GA GCGTACGTGC CTCAGG | 78 |
| 997 | 1.28 | 0.96 | CCG GGGCACGTGC CCCAGG | 213 |
| 999 | 1.3 | 0.93 | CCA TGGCACGTGC CTCAGG | 214 |
| 851 | 1.41 | 0.97 | CG GAGTACGTGA CGGAGC | 84 |
| 861 | 2.18 | 0.97 | C TAATACGTGC CGCTG | 94 |
| 809 | 4.57 | 0.97 | AGGG CCGGACGTGG GGCCCC | 42 |
| 849 | 2.66 | 0.92 | GC CTACACGTGG GTTCCC | 82 |
| 817 | 4.81 | 0.98 | CT GCCGACGTGC GCTCCGGAG | 50 |
| 823 | 4.4 | 0.94 | T GCATACGTGG GCTCCAACAG | 56 |
| 843 | 1.7 | 0.94 | GT GCATACGTGG GCTCCA | 76 |
| 901 | 1.6 | 0.94 | CACGT GCATACGTGG GCTCCA | 165 |
| 903 | 1.94 | 0.94 | CACCT GCATACGTGG GCTCCA | 166 |
| 853 | 1.73 | 0.98 | TT GCTTACGTGC GCCCGG | 86 |
| 811 | 3.9 | 0.88 | ACGCT GAGTGCGTGC GGGAC | 44 |
| 879 | 1.4 | 0.98 | GGCTGCT GCAGACGTGC AGGTC | 112 |
| 891 | 1.21 | 0.98 | GGCTGCT GCATACGTGC AGGTC | 124 |
| 887 | 1.89 | 0.98 | GGCTGCA CCGTACGTGC TGATC | 120 |
| 881 | 3.62 | 0.93 | GGCTGCA GGAGACGTGG AGAA | 114 |
| 875 | 2.63 | 0.98 | T GCAGACGTGC AGGTC | 108 |
| 885 | 4.46 | 0.96 | T ACAGACGTGC AGGTC | 118 |
| 889 | 4.14 | 0.98 | T GCATACGTGC AGGTC | 122 |
| 825 | 5.1 | 0.97 | A GGAGACGTGC GAGAA | 58 |
| 863 | 2.15 | 0.98 | A GCAGACGTGC AGGAT | 96 |
| 869 | 2.28 | 0.98 | A GCAGACGTGC AGGGT | 102 |
| 865 | 2.56 | 0.98 | A GCAGACGTGC AGGCA | 98 |
| 883 | 4.37 | 0.98 | A GAAGACGTGC AGGAT | 116 |
| 827 | 4.48 | 0.98 | A GGTTACGTGC GGACA | 60 |
| 829 | 5.12 | 0.98 | A GGAGACGTGC TGCCT | 62 |
| 837 | 3.74 | 0.97 | A GGCAACGTGC AGCCG | 70 |
| 839 | 3 | 0.63 | A GGCAATACGC AGCCG | 72 |
| 917 | 4.03 | 0.62 | CTAAGC GAGTAGCGAG TAGCC | 173 |
| 815 | 4.43 | 0.96 | G TGAGACGTGC GGCTTCCGTTTG | 48 |
| 841 | 0.99 | 0.98 | A GCGGACGTGC AGAAGTTGCACGTCCTCT | 74 |
| 909 | 1.72 | 0.98 | A GCGGACGTGC AGATGCACGTCCTCT | 169 |
| 857 | 2.46 | 1 | GCGGACGTGC GGGAACCCACGTGTAGG | 90 |
| 831 | 3.36 | 0.97 | TC CAATACGTGC AGTACT | 64 |
| 833 | 3.05 | 0.85 | TCCAATGCGT GCAGTA CT | 66 |
| 919 | 4.74 | 0.76 | CGCTACGAGC TCTACTCCAGG | 174 |
| 921 | 4.6 | 0.76 | CG CTCGACGAGC TCTACTCCA | 175 |
| 847 | 3.02 | 0.88 | GG AACAACGTGG AATTAG | 80 |
| 819 | 1.16 | 0.98 | G AAATACGTGC GCTTTGTGTGTACGTGCAGGAA | 52 |
| 907 | 0.91 | 0.98 | G AAATACGTGC GTGTGTACGTGCAGG | 168 |
| 855 | 1.07 | 0.98 | GT GTGTACGTGC AGGAAA | 88 |
| 803 | 2.56 | 0.44 | CTGTCC TCCGACTGCA TG | 36 |
| 913 | 4.88 | 0.6 | CAGGCTCC GACTACGGCT GAG | 171 |

The Initial Screening Using TransAM Kit

To assess the relative affinities of oligonucleotides for a HIF-1α containing complex, the HIF-1 TransAM assay (Active Motif, Catalog # 47096) was utilized. The assay was performed according to manufacturer's instructions. Briefly, a double-stranded oligonucletide containing the hypoxia response element (HRE) was immobilized on a 96-well plate. A nuclear extract containing HIF-1α complexes was incubated and allowed to bind to the immobilized oligonucleotide. The unbound material was washed away and the bound HIF-1α detected using an antibody that specificially recognizes HIF-1α. The anti-HIF-1α antibody was detected by a secondary antibody labeled with horseradish peroxidase (HRP), and the amount of HRP in each well was measured using a calorimetric substrate reaction and read using a microplate spectrophotometer.

The ability of candidate decoy molecules to compete for binding of HIF-1α to the HRE element-immobilized on the plate were measured and compared to reveal relative binding affinities. Candidate decoys were added in increasing molar ratios (relative to the amount of oligo immobilized on the plate) to compete for binding to the HIF-1α containing complexes. The amounts of decoys added to the assay included 0.625, 1.25, 2.5, 5, 10 and 20 fold molar excess. A well containing a competing decoy able to bind HIF-1α with high affinity would give a lower absorbance reading as compared to a decoy with low affinity for HIF-1α. All potential decoys were then compared and ranked in order to assess their relative binding affinities.

The Analysis of TransAM Result

The screen was conducted using different decoy concentrations. For each UV absorbance reading, normalization was done by calculating the ratio of absorbance readings of sample vs. wild type control. The results are summarized in Table 3. The bigger ratio represents less competition of binding with HIF-1α when compared with wild-type control. The smaller (smaller than 1.0 or close to 1.0) ratios represent better binding or better competition.

TABLE 3

| | | | |
|---|---|---|---|
| ID | Ratio | Forward Sequences | SEQ ID NO |

| | | | |
|---|---|---|---|
| 801/802 | 1.83 | GCC CTA CGT GCT GTC TCA | 34 |
| 803/804_scramble | 2.19 | CTG TCC TCC GAC TGC ATG | 36 |
| 805/806 | 1.09 | CCC CCT CGG ACG TGA CTC GGA CCA C | 38 |
| 807/808 | 2.38 | TCT GTA CGT GAC CAC ACT CAC CTC | 40 |
| 809/810 | 2.60 | AGG GCC GGA CGT GGG GCC CC | 42 |
| 811/812 | 2.37 | ACG CTG AGT GCG TGC GGG AC | 44 |
| 813/814 | 2.88 | GCC CTA CGT GCT GTC TCA CAC AGC | 46 |
| 815/816 | 2.58 | GTG AGA CGT GCG GCT TCC GTT TG | 48 |
| 817/818 | 3.40 | CTG CCG ACG TGC GCT CCG GAG | 50 |
| 819/820_double | 0.88 | GAA ATA CGT GCG CTT TGT GTG TAC GTG CAG GAA | 52 |
| 821/822 | 1.26 | CGC GAG CGT ACG TGC CTC AGG | 54 |
| 823/824 | 2.76 | TGC ATA CGT GGG CTC CAA CAG | 56 |
| 825/826 | 2.91 | AGG AGA CGT GCG AGA A | 58 |
| 827/828 | 2.62 | AGG TTA CGT GCG GAC A | 60 |
| 829/830 | 2.81 | AGG AGA CGT GCT GCC T | 62 |
| 831/832 | 2.26 | TCC AAT ACG TGC AGT ACT | 64 |
| 833/834 | 2.48 | TCC AAT GCG TGC AGT ACT | 66 |
| 835/836 | 2.51 | GGC CAG ACG TGC CAC CGG | 68 |
| 837/838 | 2.46 | AGG CAA CGT GCA GCC G | 70 |
| 839/840_mutation | 2.53 | AGG CAA TAC GCA GCC G | 72 |
| 841/842_double | 0.95 | AGC GGA CGT GCA GAA GTT GCA CGT CCT CT | 74 |
| 843/844 | 1.81 | GTG CAT ACG TGG GCT CCA | 76 |
| 845/846 | 1.57 | GAG CGT ACG TGC CTC AGG | 78 |
| 847/848 | 2.23 | GGA ACA ACG TGG AAT TAG | 80 |
| 849/850 | 1.96 | GCC TAC ACG TGG GTT CCC | 82 |
| 851/852 | 1.08 | CGG AGT ACG TGA CGG AGC | 84 |
| 853/854 | 1.62 | TTG CTT ACG TGC GCC CGG | 86 |
| 855/856 | 1.21 | GTG TGT ACG TGC AGG AAA | 88 |
| 857/858_double | 2.30 | GCG GAC GTG CGG GAA CCC ACG TGT AGG | 90 |
| 859/860 | 1.85 | ACC GTA CGT GCT GAT C | 92 |
| 861/862 | 2.50 | CTA ATA CGT GCC GCT G | 94 |
| 863/864 | 2.02 | AGC AGA CGT GCA GGA T | 96 |
| 865/866 | 2.01 | AGC AGA CGT GCA GGC A | 98 |
| 867/868 | 2.27 | TCC GTA CGT GCT GCA C | 100 |
| 869/870 | 1.93 | AGC AGA CGT GCA GGG T | 102 |
| 871/872 | 1.87 | ACC GTA CGT GCT GCC A | 104 |
| 873/874 | 2.04 | TCC GTA CGT GCT GCG T | 106 |
| 875/876 | 2.11 | TGC AGA CGT GCA GGT C | 108 |
| 877/878 | 2.29 | ACC GTA CGT GCT GCT A | 110 |
| 879/880 | 1.98 | GGC TGC TGC AGA CGT GCA GGT C | 112 |
| 881/882 | 3.39 | GGC TGC AGG AGA CGT GGA GAA | 114 |
| 883/884 | 3.76 | AGA AGA CGT GCA GGA T | 116 |
| 885/886 | 4.02 | TAC AGA CGT GCA GGT C | 118 |
| 887/888 | 1.94 | GGC TGC ACC GTA CGT GCT GAT C | 120 |
| 889/890 | 2.90 | TGC ATA CGT GCA GGT C | 122 |
| 891/892 | 1.60 | GGC TGC TGC ATA CGT GCA GGT C | 124 |

The central core and the 5′ and 3′ flanking sequences are numbered as follows:
−4−3−2−1 1 2 3 4 5+1+2+3
where the numbering of the core sequence is highlighted, the 5′ flank sequences are labeled with negative numbers, and the 3′ flank sequences are labeled with positive numbers.

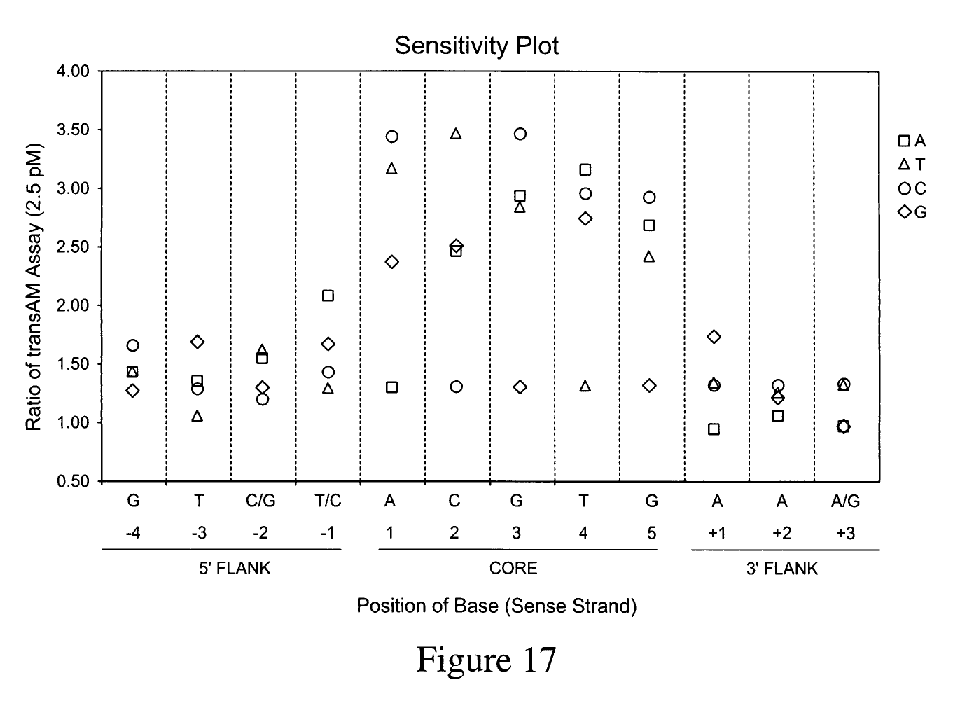

FIG. 17 is a sensitivity plot, displaying the effect of various nucleotide base substitutions at positions −4 through +3 of the sense strand on the binding affinity of a HIF oligonucleotide decoy molecule.

(1) The data shows that the core sequence (positions 1-5) must be ACGTG (SEQ ID NO: 126) for maximum binding affinity. The excellent binding affinity of decoys with “A” at position +1 is a significant new and unexpected finding.

(2) Another important finding is that having “G” at position +1 significantly decreases binding affinity, and should, therefore, be avoided.

(3) Decoys having “A” at position −1 have also showed reduced binding.

(4) Decoys having T or A at position −2 showed reduced binding.

(5) Decoys having “T” at position −3 have excellent binding affinity.

(6) Decoys having A or G at positions +5 have increased affinity.

(7) This data also clearly shows that there are no special requirements for base composition at positions −4 or +2, therefore, the decoy molecules herein can contain any base at these positions.

(8) Comparison of the immediate 5′ sequences suggests that the base composition of GCAG or GGAG or GCAT or CCCT or CCGT could lead to poor competition (e.g., bigger ratio, compared with wild type decoy).

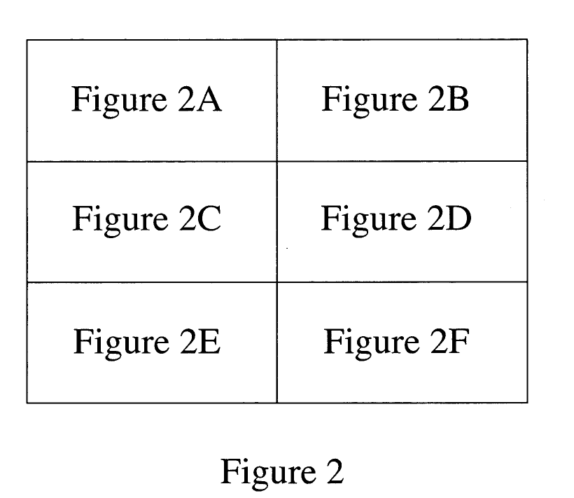

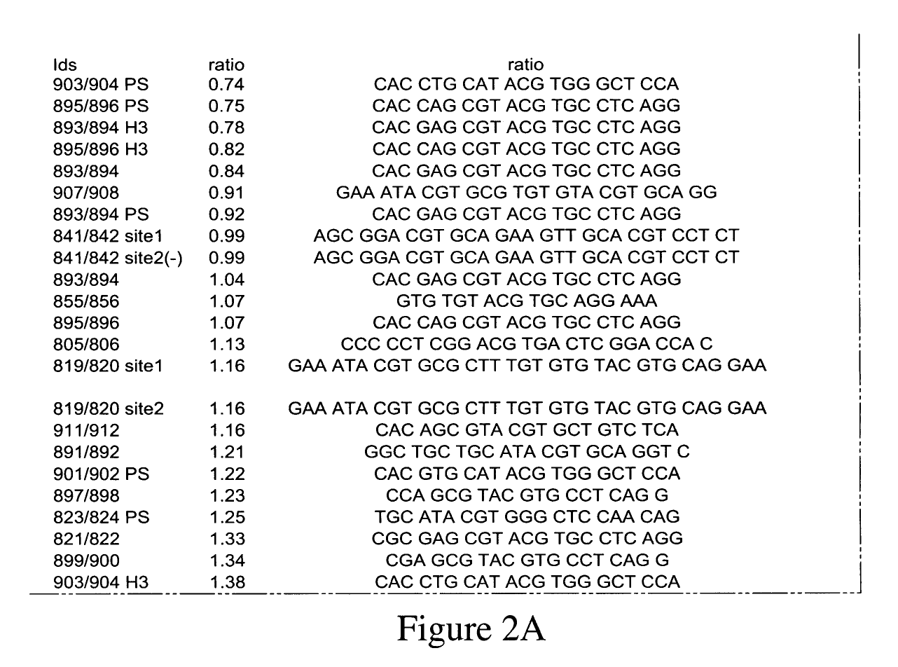

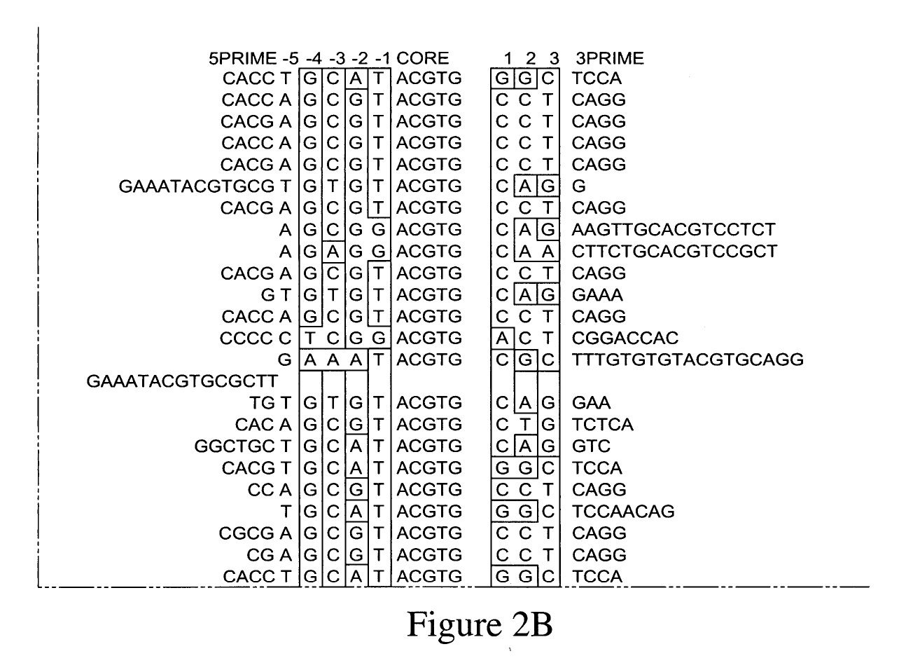

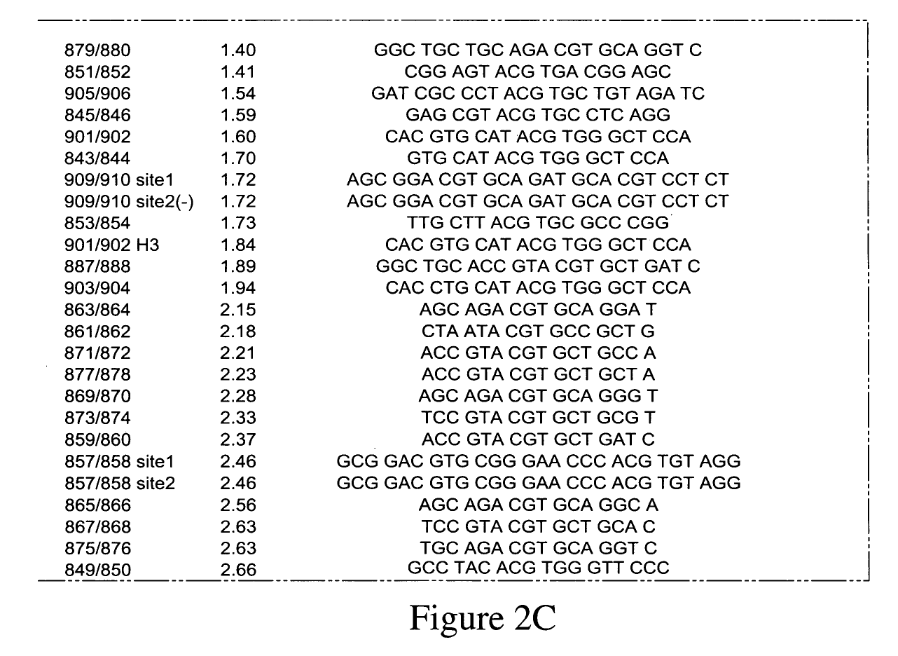

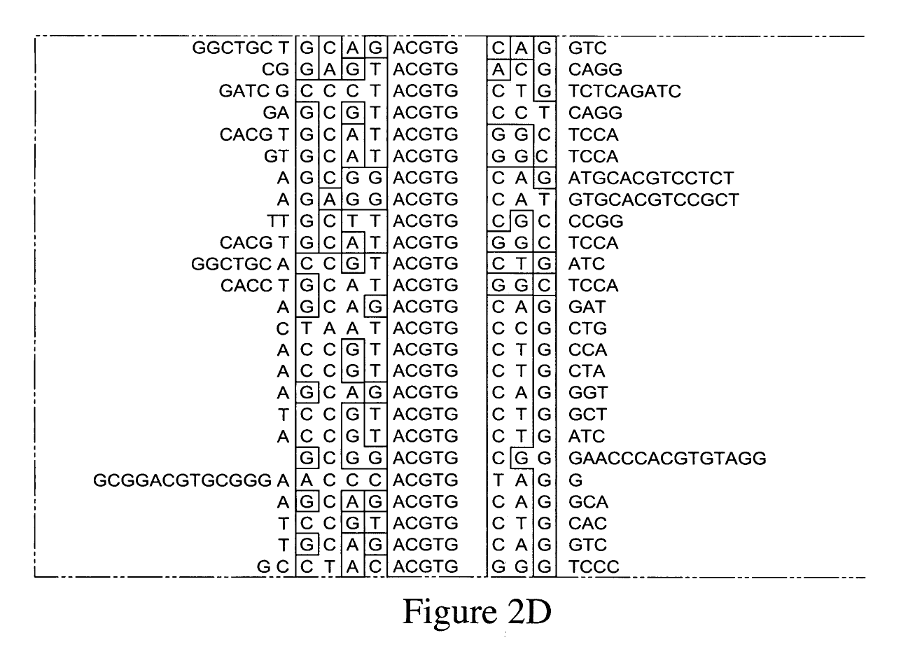

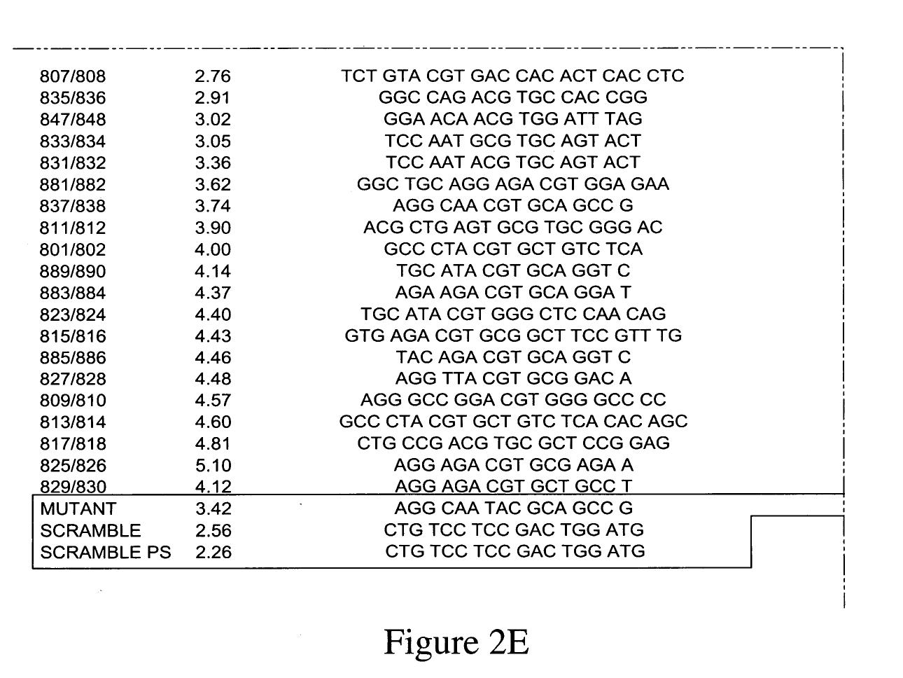

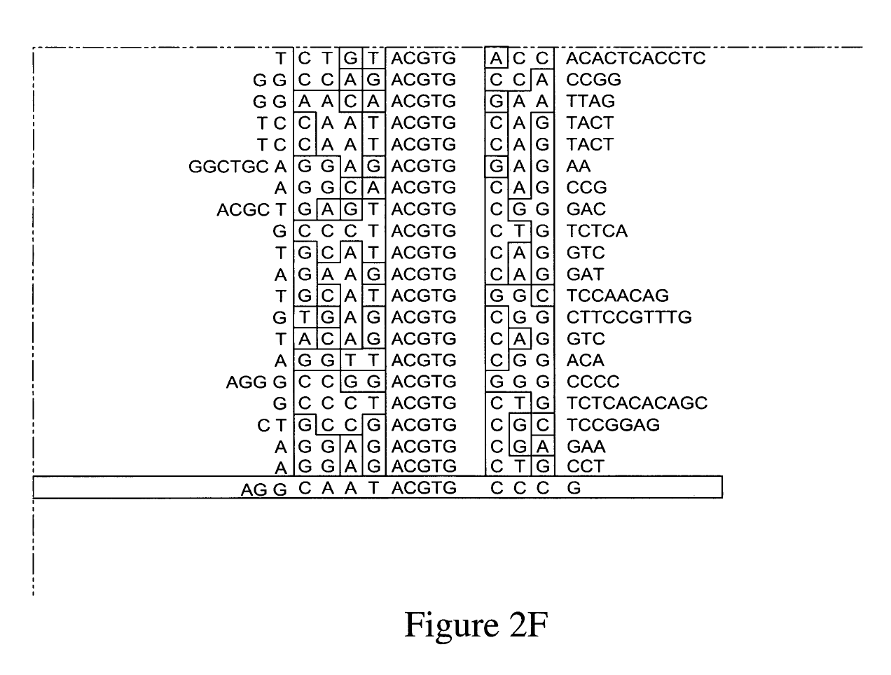

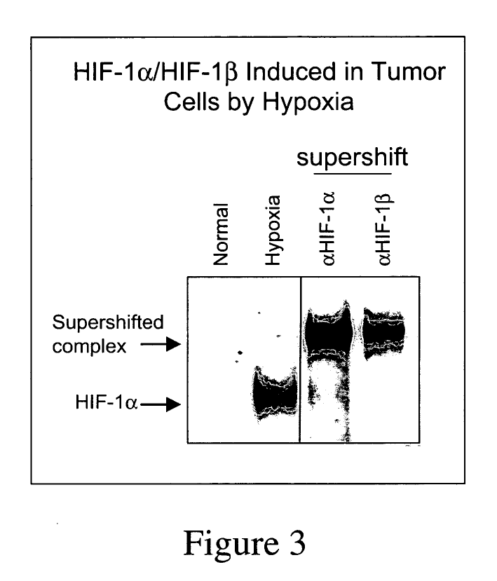

(9) If we sort the ratio, those decoys with better competition (e.g., smaller ratio) mostly share base “G” and base “T” at position “−4” and “−1” respectively (FIG. 3). The 4 bases immediately before core (ACGTG; SEQ ID NO: 126) will be more like “GCGT” (SEQ ID NO: 127) for the better competition decoys (FIG. 2). FIG. 2 also suggests that the combination of “G” at position “−4” with “G” at position “−1” does not favor the binding affinity, same to the combination of “A” at position “−3” and “A” at position “−2”, respectively.

Confirmation by EMSA

The HIF gel shift assays (EMSA) were performed as follows. A double-stranded oligonucleotide containing a consensus HIF binding site was end-labeled with γ³²P-ATP using T4 Polynucleotide Kinase (Promega). One microgram of a nuclear extract prepared from LPS stimulated THP-1 cells (human monocyte cell line) was incubated with 35 fmol of radiolabeled probe in the presence or absence of competing unlabeled HIF double-stranded oligonucleotides (dsODN) or scrambled dsODN. The incubations were carried out at room temperature for 30 minutes in a 20 μl reaction volume composed of 10 mM Tris-HCl pH 8, 100 mM KCL, 5 mM MgCl2, 2 mM DTT, 10% Glycerol, 0.1% NP-40, 0.025% BSA and 1 μg Poly-dIdC. The reactions were loaded onto a 6% polyacrylamide gel, subjected to electrophoresis and dried. The dried gels were imaged and quantitated using a Typhoon 8600 Phosphorlmager (Amersham) and ImageQuant software. The identity of the HIF proteins contained in complexes bound to the radiolabeled oligonucleotide probe were identified by pre-incubating the reactions for 5 minutes with individual antibodies specific for each member of the HIF family prior to the addition of the radiolabeled probe.

The binding of selected decoys is confirmed by conventional EMSA method.

FIG. 18 illustrates the relationship between the predicted binding and observed competition ratio. If the bioinformatics approach accurately predicted the ability of any given decoy to bind to HIF, such as HIF-1, the plot of the predicted vs actual data would be a straight line, with an excellent correlation coefficient. As FIG. 18 illustrates, however, there is a relatively poor correlation between the predicted and actual binding/competition of the decoy molecules. In FIG. 18, the sequences are divided into several categories. The natural sequences from HIF-1-regulated genes are shown as circles; the designed decoy sequences are shown as diamonds; sequences specifically designed to test specific structure activity relationship (SAR) questions are triangles; and the squares represent control or mutant sequences that were intended to be poor binders.

A few striking examples are sequence 857 with a predicted binding score of 0.997, which has an actual score above 3 at 2.5 fold molar excess. Sequence 8.59 has a predicted score of 0.978 and an actual score of 2.42 at 2.5 fold molar excess. Comparison of the best binders (below a ratio of 1) has predicted scores from as low as 0.938 to almost 1 (0.99).

The poor correlation between the predicted and absolute scores underscore the necessity of actual structure-function studies, including analysis of the effect of the length and composition of the flanking sequences, in the design of HIF decoy molecules.

### EXAMPLE 2

The Effect of the Length of 5′ Flank Sequences on Binding Properties

Table 4 below shows the comparison of a series of decoy molecules that all include the optimal core and a known good 3′ flank sequence. The key difference among these sequences is the length of the 5′ flank sequence. A large number of additional decoys with a 5′ flank of 7 or more bases were also analyzed, and those with the optimal core and a good 3′ flank all were found to have scores (competition ratios) in the 1.25 or better range. Thus, a 5′ flank of 5 or fewer bases is generally not sufficient to support good HIF binding. a 3′ flank with 6 bases may show good binding, but sequences with more than 7 bases in the 3′ flank region generally have much better binding properties. The results of this study also suggest that there is a preference for A at position +1 in the 3′ flanking sequence, while G is not favored at the position. In addition, there is a preference for a higher GC content in the 3′ flanking sequence.

TABLE 4

| | Competition | |
|---|---|---|
| | ratio at 10 fold | Length of 5′ |
| Sequence | molar excess | flank (bases) |
| 859 | 2.42 | 5 |
| 867 | 2.92 | 5 |
| 873 | 3.10 | 5 |
| 871 | 2.59 | 5 |
| 877 | 2.62 | 5 |
| 801 | 9.75 | 5 |
| 813 | 7.81 | 6 |
| 835 | 3.74 | 6 |
| 845 | 1.34 | 6 |
| 851 | 1.23 | 6 |
| 999 | 1.36 | 7 |
| 963 | 1.13 | 7 |
| 899 | 1.20 | 7 |
| 911 | 1.42 | 8 |
| 905 | 1.52 | 9 |

### EXAMPLE 3

The Effect of Backbone Substitutions on Binding Affinity

A series of experiments were performed comparing the binding affinity of a single decoy sequence (895/896) with no sulfur substitutions in phosphodiester linkage of the backbone (PO), to those with up to six sulfur substitutions in the phosphodiester bond starting from the 3′ end. These labeled H, for hybrid backbone, and with the number of substitutions starting from the 3′ end. For example, H3 designates a hybrid backbone with substitutions at positions linkage 1, 2 and 3, starting from the 3′ end. If all phosphodiester linkages are substituted, the molecule is designated PS.

TABLE 5

| | | Ratio at 0.625 fold | Ratio at 2.5 fold |
|---|---|---|---|
| 895 | 896 | molar excess | molar excess |
| PO | PO | 0.94 | 1.22 |
| H1 | H1 | 1.98 | 2.01 |
| H2 | H2 | 0.90 | 0.86 |
| H3 | H3 | 1.06 | 1.33 |
| H4 | H4 | 1.12 | 1.42 |
| H5 | H5 | 1.18 | 1.34 |
| H6 | H6 | 0.98 | 1.01 |
| H3 | PS | 1.35 | 1.38 |
| PS | PS | 0.95 | 0.98 |
| PS | PO | 0.74 | 0.52 |
| H5 | PO | 1.18 | 1.27 |

The data listed in Table 5 shows that, compared to fully phosphodiester backbone 895/896 PO/PO, H2, H4, H5, and PS, as well as mixed strand H3/S, PS/PO and H5PO all maintain good binding. The only substitution that did not perform well was H1. Accordingly, the decoys of the present invention include decoys with modified backbones.

### EXAMPLE 4

The HIF Decoy Molecule Binds to the HIF-1α/HIF-1β Complex

Methods

The HIF-1α gel shift assays were performed as follows. A double-stranded oligonucleotides (Sigma Genosys) containing the HIF-1α binding site for the HIF-1α Decoy (5′CACCAGCGTACGTGCCTCAGG 3′ (SEQ ID NO: 130) was end-labeled with γ³²P-ATP using T4 Polynucleotide Kinase (Promega). Five μg of a nuclear extract prepared from either normoxic or hypoxic MiaPaCa (pancreatic tumor cell line) was incubated with 35 fmol of radiolabeled probe in the presence or absence of antibodies specific to either HIF-1α or HIF-1\tilde{β} The incubations were carried out at room temperature for 30 minutes in a 20 μl reaction volume composed of 25 mM Tris pH 7.6, 100 mM KCL, 0.5 mM EDTA, 1 mM DTT, 10% Glycerol, 0.2M PMSF, 0.2M sodium orthovanadate and 1 μg Poly-dIdC (Roche). The reactions were loaded onto a 5% polyacrylamide gel, subjected to electrophoresis and dried. The dried gels were imaged and quantitated using a Typhoon 8600 Phosphorimager (Amersham) and ImageQuant software. The identity of the HIF-1α proteins contained in complexes bound to the radiolabeled oligonucleotide probe were identified by pre-incubating the reactions for 5 minutes with individual antibodies specific for each member of the HIF-1α family prior to the addition of the radiolabeled probe.

Results

When exposed to hypoxia, a protein complex is induced which binds to the HIFα radiolabeled probe. As shown in FIG. 3, antibodies against both HIF-1α and HIF-1β were able to supershift the band, indicating that the antibodies bind specifically to their target therefore slowing the mobility of the complex. This indicates that this band is composed of a HIF-1α/HIF-1β heterodimer.

In all examples below, the HIF decoy molecule is HIF decoy 895:896H3 upper strand-CAC CAG CGT ACG TGC CTC*A*G*G (SEQ ID NO: 134): complementary strand-CCT GAG GCA CGT ACG CTG*G*T*G (SEQ ID NO: 135).

### EXAMPLE 5

HIF Decoy Binds and Blocks HIF but does not Inhibit other TFs

Methods

The ability of HIF Decoy 895:896H3 to bind and therefore block activity of the target, HIF-1, as well as other non-target TFs was determined by TransAM™ method plate assays (Active Motif, Carlesbad, Calif. 92008), using nuclear extracts from the hypoxia-induced cells described in Example 4.

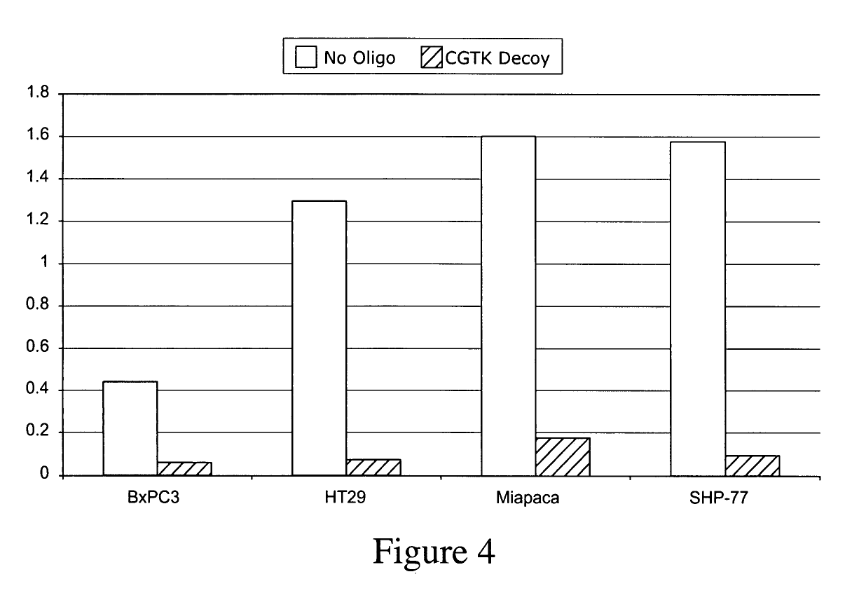

Briefly, oligonucleotide containing the HIF-1 binding site from the erythropoietin (EPO) promoter region was immobilized on a 96 well plate. Nuclear extracts (5 micrograms) from hypoxia-induced BxPC3, HT29, MiaPaca and SHP-77 cells were added to the wells in the presence or absence of a 10-fold molar excess of HIF Decoy (895:896H3) and incubated to allow the HIF-1 to bind to the immobilized EPO binding site. Following a wash step, the amount of HIF-1 bound to the plate was measured by incubating using an antibody specific for HIF-1α, followed by a secondary HRP-conjugated antibody to detect the anti-HIF-1α antibody. The amount of peroxidase was measured spectroscopically. The amount of binding in the absence of decoy represents the maximum HIF-1 binding in the extract. The reduction in binding in the presence of the decoy is used to measure the ability of the decoy to compete for HIF-linding. The results are shown in FIG. 4.

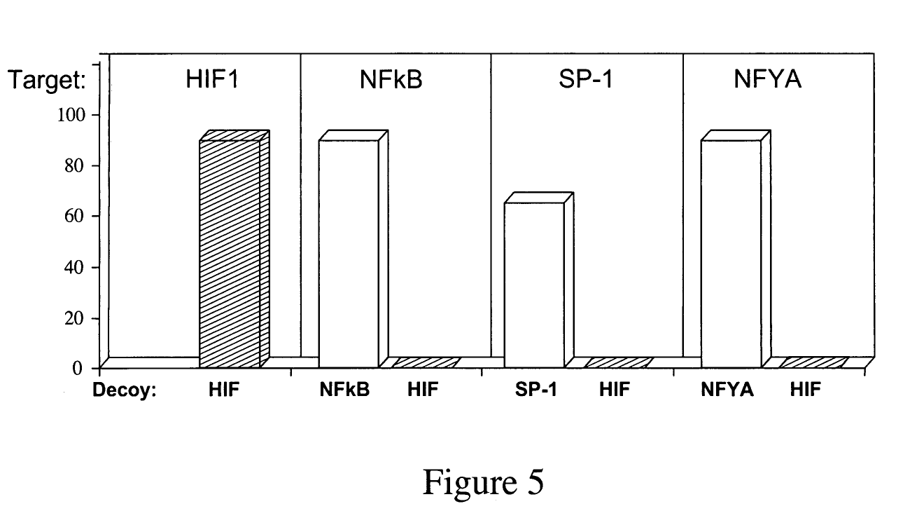

Similar assays were performed using TransAM™ kits specific for the non-target transcription factors, NF-κB, SP-1, and HFYA. All assays were performed following the manufacturer's instructions with the addition of completing HIF decoy 895:H3 at a 10× molar access (compared to the immobilized oligonucleotide). The results are shown in FIG. 5.

Results

HIF Decoy 895:896H3 was able to compete with the immobilized EPO promoter binding site for HIF binding in nuclear extracts for all four cell lines tested. As shown in FIG. 5, decoys to the target TF were able to compete for binding to the immobilized target binding site whereas the HIF decoy was not able to block binding of any of these non-target transcription factors.

### EXAMPLE 6

HIF Decoy Completes for Binding HIF-1α/HIF-1β to Two Natural Promoters

The objective of this study was to show that a HIF-1α decoy is capable to compete for binding of the HIF-1α/HIF-1β complex from two natural promoters, erythropoietin (EPO) and the transferrin receptor, using gel shift assay.

Methods

The HIF-1α gel shift assays were performed as follows. Double-stranded oligonucleotides (Sigma Genosys) containing the HIFα binding site from the Transferrin Receptor (5′CGCGAGCGTACGTGCCTCAGG 3′; SEQ ID NO: 131) or that contained in the Erythropoietin (EPO) promoter (5′ GCCCTACGTGCTGTCTCA 3′; SEQ ID NO: 132) were end-labeled with γ32P-ATP using T4 Polynucleotide Kinase (Promega). Five μg of a nuclear extract prepared from hypoxic SHP-77 cells (small cell lung carcinoma tumor cell line) was incubated with 35 fmol of the radiolabeled probe in the presence or absence of increasing molar amounts of competing unlabeled HIFα double-stranded oligonucleotide Decoy (ODN). The incubations were carried out at room temperature for 30 minutes in a 20 μl reaction volume composed of 25 mM Tris pH 7.6, 100 mM KCL, 0.5 mM EDTA, 1 mM DTT, 10% Glycerol, 0.2M PMSF, 0.2M sodium orthovanadate and 1 μg Poly-dIdC (Roche). The reactions were loaded onto a 5% polyacrylamide gel, subjected to electrophoresis and dried. The dried gels were imaged and quantitated using a Typhoon 8600 Phosphorlmager (Amersham) and ImageQuant software. The identity of the HIFα proteins contained in complexes bound to the radiolabeled oligonucleotide probe had been previously identified by pre-incubating the reactions for 5 minutes with individual antibodies specific for each member of the HIFα family prior to the addition of the radiolabeled probe (data not shown).

Results

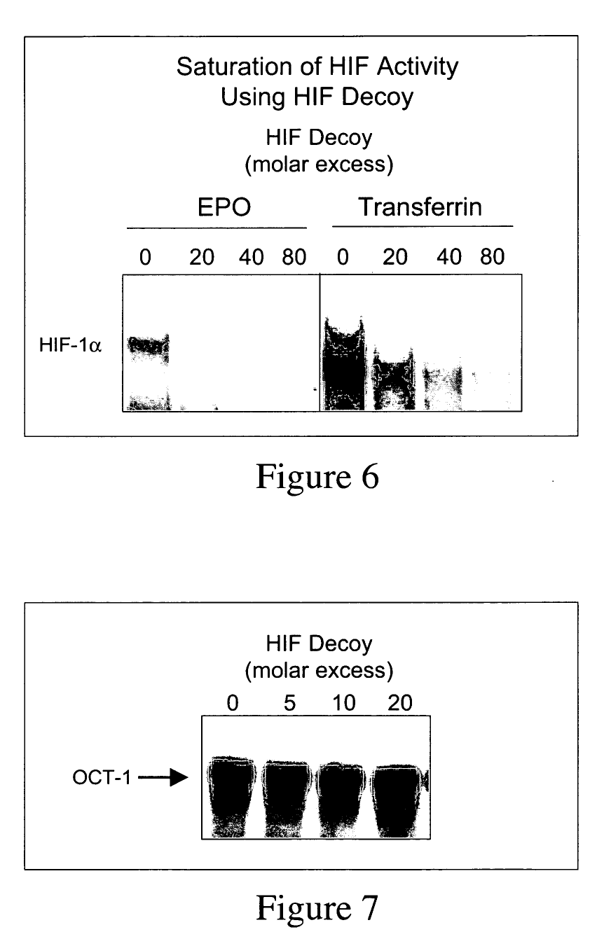

As shown in FIG. 6, the HIF-1α decoy was able to compete effectively for the binding of HIFα from two natural promoters tested. In the case of the EPO promoter, the HIFα decoy was able to effectively compete for binding of the HIF-1α/HIF-1β complex at 20-fold molar excess (lower concentrations not tested at this point). With the transferrin receptor promoter, the HIF-1α decoy was able to effectively compete for binding of most of the HIF-1α/HIF-1β complex at 20-fold molar excess.

### CONCLUSIONS

It was possible to induce tumor cells to express high levels of the HIF-1α transcription factor when exposed to hypoxic conditions and identify the complex using the gel shift assay. The HIF-1α decoy was able to compete for binding of the HIF-1α/HIF-1β complex away from the HIF-1α binding sites from two natural promoters, erythropoietin and transferrin receptor.

### EXAMPLE 7

HIF Decoy does not Bind Transcription Factor Oct-1

Studies were performed to show that the HIF-1α Decoy does not bind to the ubiquitous transcription factor, Oct-1 using electrophoresis mobility shift assay (EMSA), also called a gel shift assay.

Methods

The Oct-1 gel shift assay was performed as follows. A double-stranded oligonucleotide (Promega) containing the Oct-1 binding site (5′ TGTCGAATG CAAATCACTAGAA 3′; SEQ ID NO: 133) was end-labeled with γ32P-ATP using T4 Polynucleotide Kinase (Promega). Five μg of a nuclear extract prepared from MiaPaCa cells was incubated with 35 fmol of radiolabeled probe in the presence or absence of increasing molar amounts of competing unlabeled HIF-1α double-stranded oligonucleotide Decoy (ODN). The incubations were carried out at room temperature for 30 minutes in a 20 μl reaction volume composed of 10 mM Tris pH 8.0, 100 mM KCL, 5 mM MgCl₂, 2 mM DTT, 6% Glycerol, 0.1% NP-40, 0.02% BSA and 1 ug Poly-dIdC (Roche). The reactions were loaded onto a 6% polyacrylamide gel, subjected to electrophoresis and dried. The dried gels were imaged and quantitated using a Typhoon 8600 Phosphorlmager (Amersham) and ImageQuant software. The identity of the Oct-1 proteins contained in complexes bound to the radiolabeled oligonucleotide probe was identified by competing the bound complex away with the Oct-1 oligonucleotide versus a scrambled sequence.

Results

As shown in FIG. 7, binding of an irrelevant transcription factor, Oct-1, to its specific binding site was not inhibited by HIF-1α Decoy.

### EXAMPLE 8

Treatment of Cancer Cells with HIF Decoy Induces Hypoxia-Induced HIF Activity

Methods

HT-29 (human colon carcinoma), MiaPaCa2 and BxPc3 (human pancreatic carcinoma) and SHP-77 (NSCLC) tumor cell lines were obtained from ATCC and were maintained in 5% Co2 in appropriate media. HIF activity was induced by incubating the cells in 1% O₂ conditions for up to 24 hours or by the addition of 260 μM CoCl₂ to the media as reported by Behrooz and Ismail-Beigi (J. Biol. Chem. 133:151-60 (1997)). In order to measure the ability of the HIF decoy to block HIF activity in these cells, the cells were transfection with various amounts of HIF-1 Decoy 895:896H3 using 10 min of pressure treatment at 6 psi. Nuclear extracts were prepared from the cells 24 hours after addition of the Decoy.

The amount of active HIF-1 in nuclear extracts was quantified using gel shift assays. A double-stranded oligonucleotide (Sigma Genosys) containing the HIFα binding site for the HIFα Decoy (5′CACCAGCGTACGTGCCTCAGG 3′; SEQ ID NO: 130) was end-labeled with γ32P-ATP using T4 Polynucleotide Kinase (Promega). Five μg of a nuclear extract prepared from either normoxic or hypoxic MiaPaCa (pancreatic), SHP-77 (small cell lung carcinoma), HT-29 (colon) or BxPc-3 (pancreatic) tumor cells was incubated with 35 fmol of radiolabeled probe. The incubations were carried out at room temperature for 30 minutes in a 20 μl reaction volume composed of 25 mM Tris pH 7.6, 100 mM KCL, 0.5 mM EDTA, 1 mM DTT, 10% Glycerol, 0.2M PMSF, 0.2M sodium orthovanadate and 1 ug Poly-dIdC (Roche). The reactions were loaded onto a 5% polyacrylamide gel, subjected to electrophoresis and dried. The dried gels were imaged and quantitated using a Typhoon 8600 Phosphorlmager (Amersham) and ImageQuant software. The identity of the HIF-1α proteins contained in complexes bound to the radiolabeled oligonucleotide probe were identified by pre-incubating the reactions for 5 minutes with individual antibodies specific for each member of the HIF-1α family prior to the addition of the radiolabeled probe.

The amount of huVEGF secreted into the media was measured using a huVEGF Quantikine ELISA kit exactly as described by the manufacturer (R&D systems, Minneapolis, Minn. 55413). The cells were harvested, mRNA prepared using an RNAeasy™ 96 well kit (Qiagen Inc. 27220 Tumberry Lane, Valencia, Calif. 91355) again exactly as described by the manufacturer. The amount of VEGF mRNA was quantified relative to the amount of β-actin mRNA using quantitative PCR in an ABI-Prism-7900HT cycler with ABI SDS 2.2 software as per the manufactures instructions.

Results

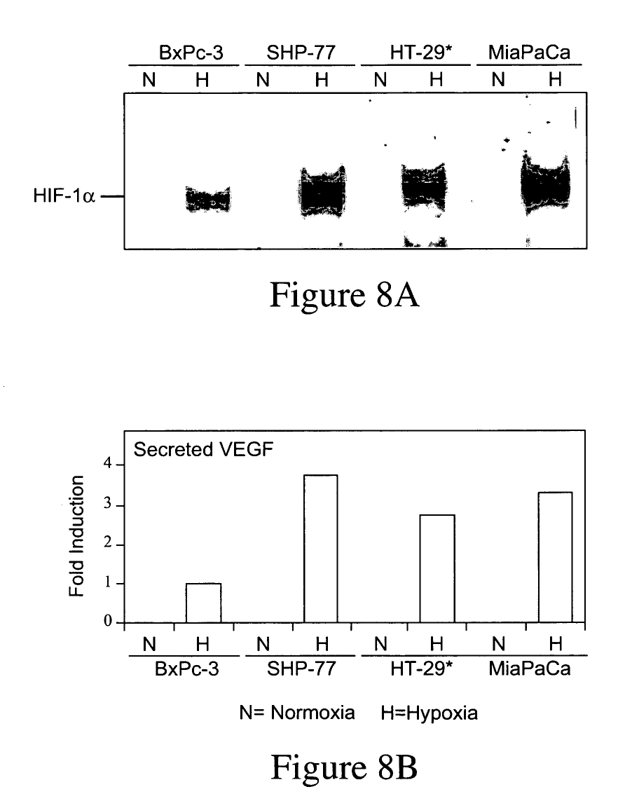

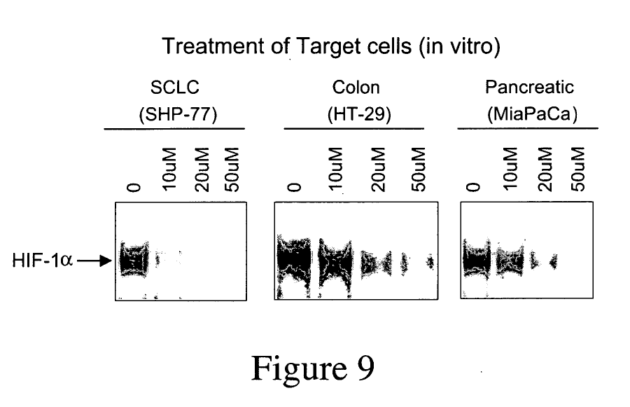

HIF-1α activity, measured by gel shift, and secreted VEGF, measured by ELISA, were increased in all cell lines by hypoxia (FIGS. 8A and B).

Transfection of the tumor cells with increasing concentrations of HIF Decoy 895:896H3 reduced HIF-1 binding to a HIF-1 consensus binding site (5′ CACCAGCGTACGTGCCTCAGG 3′, SEQ ID NO: 130) in gel shift assays as shown in FIG. 7.

### EXAMPLE 9

Efficacy of HIF Decoy in Xenograft Studies

Xenograft Tumor Models

6-8 week old nu/nu mice were implanted subcutaneously with human tumor cell lines. When the tumors reach 150-250 mm3 volumes they are randomized into groups of 6 to 15, such that each group has an equivalent mean volume, and animals are treated either by continuous subcutaneous delivery via Alzet osmotic mini-pump inserted dorsally, or by bolus ip or iv injection. All decoys were re-suspended in saline and appropriate vehicle controls were included in every study.

On the day of implantation cells were harvested, rinsed twice in culture media without FBS, counted, and appropriately diluted to obtain a suspension of 50-100 million cells per ml (if necessary cells were diluted 1:2 with 50% Matrigel just before implantation). Mice received subcutaneous injection of cells 3-5×106 cells in the ventral side of the abdomen just off the midline. All mice were weighed and caliper measurements taken every 3->7 days after tumors became palpable and able to be measured using the caliper. The length and width was used to calculate the measured tumor volume. Tumor volume was determined using the formula (V=(length×(width)2/2).

Tumor Analysis

At the end of each experiment (1-6 weeks after treatment initiation) animals were euthanased by exanguination under anaesthesia and tumors (and other tissues) from each group colleted weighed and fixed in 10% neutral buffered formalin or snap frozen in liquid nitrogen. Fixed tissues were processed for histological analysis of various markers such as hypoxia, apoptosis, blood vessels (CD-31 detection), HIF-1, VEGF etc. Serum samples were analyzed for mVEGF and mEPO levels by ELISA using Quantikine kids from R&D Systems as previously described.

Efficacy Studies in HT-29 Colon Xenograft Tumors

One of the standard therapeutics for colon cancer treatment if 5-FU. A study comparing HIF decoy 895:896H3 delivered to mice carrying HT-29 tumors with 5-FU alone and in combination was carried out.

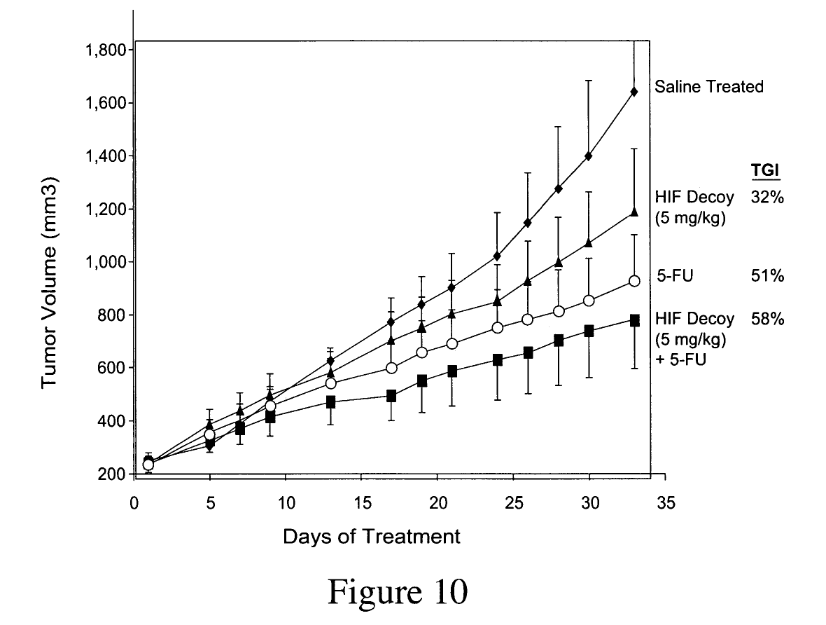

Decoy was dosed by daily ip injection at 5 mg/kg/day and 5-FU was dosed. Treatment with HIF Decoy (daily ip injection at 5 mg/kg/day) reduced the rate of tumor growth (FIG. 10). Tumor growth inhibition (TGI) was calculated using the formula==(size of treated tumors at end of treatment−size of treated tumors at beginning of treatment)/(size of vehicle tumors at end of treatment−size of vehicle tumors at beginning of treatment)−1)*100. A TGI of 51% was observed with 5-FU (2 times per week at 25 mg/kg/dose by intra-venous (i.v.) tail vein injection) and a TGI of 58% was observed with the two drugs were combined.

HIF Decoy Increases Apoptosis

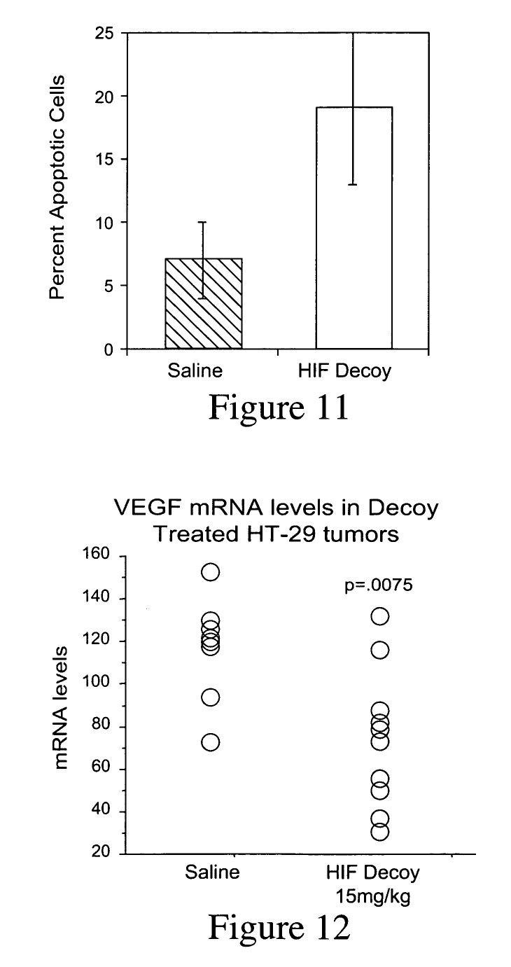

Four saline treated control tumors and 4 tumors from the HIF Decoy 895:896H3 treated group were fixed, sectioned and stained for apoptotic bodies using TumorTACS™ (Trevigen, Inc. Gaithersburg, Md. 20877) to detect fragmented chromosomal DNA using a florescent FITC label. Counter staining of nuclei was performed using Hoescht stain. Imaging of the stains was performed by taking 5 random images at 10× magnification from a central cross-section of the tumor using a Zeiss Axioskop 2 Plus microscope fitted with a SPOT digital camera (Diagnostic Inst. Inc.) Apoptosis quantification was performed using ImagePRO software. The number of nuclei present was determined by capturing the Hoescht fluorescence and the number of these nuclei that were also stained by the TumorTACS™ taken as the percentage of apoptotic cells. HIF Decoy treatment resulted in a 2.5 fold increase in the number of apoptotic cells (FIG. 11).

In a second study, mice bearing HT-29 tumors were administered HIF Decoy 895:896H3 at a dose of 15 mg/kg/day, delivered continuously by subcutaneous infusion. Tumors were frozen; mRNA prepared using standard methods, and VEGF mRNA quantified as described above. There was a significant reduction (p=0.0075 Fisher's PLSD) in the amount of VEGF mRNA in the tumors of the treated animals (FIG. 12).

### EXAMPLE 10

Combination Treatment with Avastin™

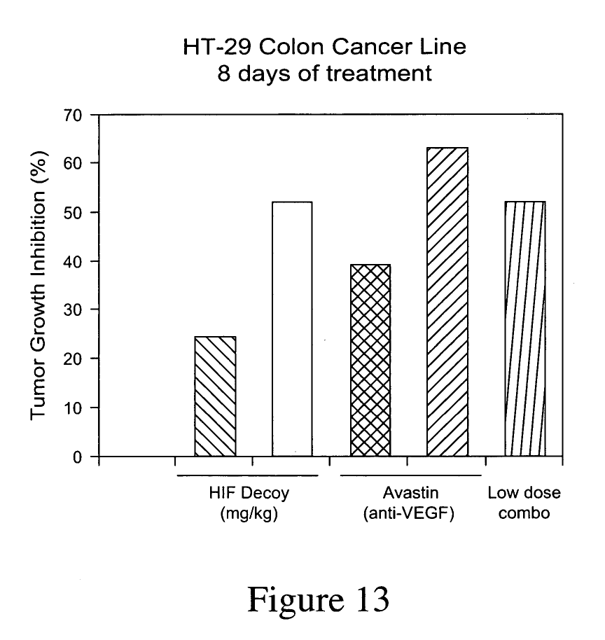

It was hypothesized that inhibition of tumor angiogenesis would make the tumor more hypoxic and increase the amount of HIF, thereby increasing the therapeutic window for HIF decoy. This would imply that combination therapy with HIF Decoy 895:896H3 and angiogenic agents would be more therapeutic. To test this hypothesis HIF Decoy 895:896H3 was administered by continuous infusion to two groups of animals at two doses (30 mg/kg/day and 45 mg/kg/day). The anti-angiogenic agent Avastin™ (anti-VEGF antibody Genentech, South San Francisco, Calif.) was delivered to two groups of mice at doses of 0.4 mg/kg/dose (low dose) and 2 mg/kg/dose (maximal dose) twice weekly by i.v. injection. In a sixth group both low dose Avastin™ and low dose HIF Decoy 895:896H3 were used. The results of this study are shown in FIG. 13. After 8 days of treatment the 30 mg/kg/day Decoy treated group showed a 24% TGI and the 45 mg/kg/day demonstrated 52% TGI. This dose was the same as the combined 30 mg/kg/day decoy plus low dose Avastin™. Alone the low dose Avastin™ resulted in 39% TGI and the high dose 63% TGI.

These data show that the HIF decoy 895:896H3 treatment gives a greater than 50% tumor inhibition at high dose of decoy. This level of inhibition is similar to that seen with Avastin™, and in this experiment combination therapy with Avastin™ had an additive effect.

### EXAMPLE 11

Efficacy Studies in Further Tumor Models

Efficacy Studies in SHP-77

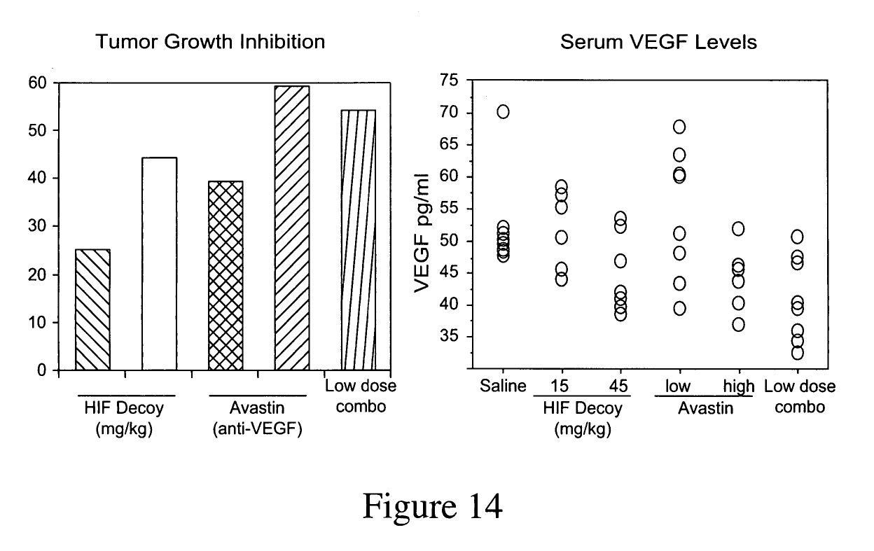

Cells were grown and implanted as described in the previous example. HIF1 decoy 895:896H3 was administered by continuous infusion to two groups of animals at two doses (30 mg/kg/day and 45 mg/kg/day). The anti-angiogenic agent Avastin™ (anti-VEGF antibody Genentech, South San Francisco, Calif.) was delivered to two groups of mice at doses of 0.4 mg/kg dose (low dose) and 2 mg/kg dose (maximal dose) twice weekly by i.v. injection. In a sixth group both low dose Avastin™ and low dose HIF decoy 895:896H3 were used. The results of this study are shown in FIG. 14. After only 7 days of treatment the effect of 45 mg/kg/day HIF Decoy 895:896H3 was equivalent or better than that seen with low dose Avastin™ and the combination group was similar to that seen with high dose Avastin™. Sera was analyzed from these mice and the VEGF levels measured. There was a significant dose dependent reduction in VEGF for all treatments and the effect was greatest in the combination treatment.

Efficacy Studies in MiaPaCa2

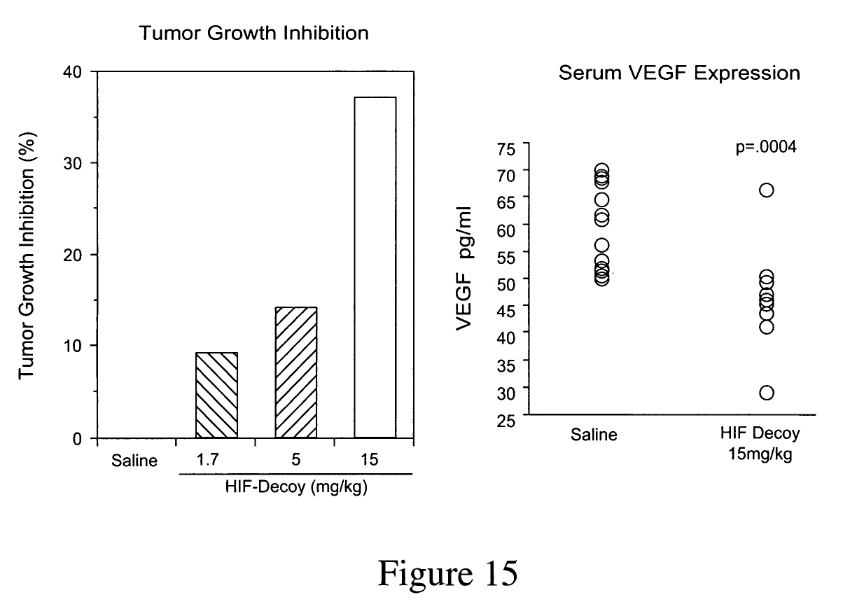

Xenograft models of MiaPaCa2 mice using matrigel were established as described. In one study where groups of animals were treated for 28 days with three doses of HIF Decoy 895:896H3 (1.7 mg/kg/day, 5 mg/kg/day and 15 mg/kg/day) there was a dose dependent increase in the TGI which was significant at the 15 mg/kg/day dose (p=0.0139 Mann-Whitney) as shown in FIG. 15, left panel.

The levels of circulating muVEGF were also measured in the 15 mg/kg/day animals and as before there was a significant reduction in these levels from those of the saline treated controls as shown in FIG. 15, right panel.

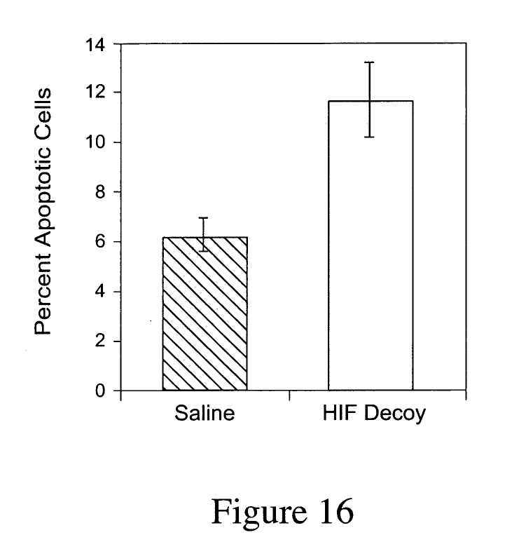

Finally size matched tumors from saline treated and 15 mg/kg/day treated animals (3 per group) were fixed, processed and stained with M30 CytoDEATH antibody as described by the manufacturer (Roche Applied Science, Normenwald 2, 82372 Penzberg, Germany). The CytoDEATH antibody specifically binds to a caspase cleaved, formalin resistant epitope of the human cytokeratin 18 (CK18) cytoskeletal protein and is a marker for cells in all stages of apoptosis. Images were captured as before and analyzed using ImageJ software from NIH. The resulting data (FIG. 16) demonstrated a significant (p=0.0299 Fisher's PLSD) increase in apoptosis in HIF Decoy 895:896H3 treated MiaPaCa2 tumors.

Based on the test set forth in the Examples above, a preferred group of HIF dsODN molecules contains a sense strand selected from the group of decoy Nos. 895, 985, 987, 963, 993, and 995.

All references cited throughout the disclosure are hereby expressly incorporated by reference.

Although the invention has been illustrated by reference to certain embodiments, it is not so limited. Based on the results presented herein, one of ordinary skill will apreciate that various further modifications are possible, and can be performed without undue experimentation, in order to design an optimal decoy of a particular application. All such modifications and alterations are within the scope herein.

1. A HIF double-stranded oligodeoxynucleotide (dsODN) molecule comprising a sense and an antisense strand, in which the sense strand comprises, in 5′ to 3′ direction, a sequence of formula FLANK1-CORE-FLANK2, wherein
CORE is the sequence ACGTG (SEQ ID NO: 126),
FLANK1, in which the nucleotide positions are designated by negative (−) numbers, is at least 6 nucleotides long, and
FLANK 2, in which the nucleotide positions are designated by positive (+) numbers, has a GC content of at least about 50%, and
wherein said dsODN molecule is capable of specific binding to HIF.

2. The dsODN molecule of claim 1 wherein FLANK2 has a nucleotide other than G at position +1.

3. The dsODN molecule of claim 1 wherein FLANK2 has the nucleotide A at position +1.

4. The dsODN molecule of claim 1 wherein FLANK2 has a nucleotide A or G at position +3.

5. The desODN molecule of claim 1 wherein FLANK2 has any nucleotide at position +2.

6. The dsODN molecule of claim 1 wherein FLANK1 has a nucleotide other than A at position −1.

7. The dsODN molecule of claim 1 wherein FLANK1 has a nucleotide T or C at position −1.

8. The dsODN molecule of claim 1 wherein FLANK1 has a nucleotide other than G at position −3.

9. The dsODN molecule of claim 1 wherein FLANK1 has the nucleotide T at position −3.

10. The dsODN molecule of claim 1 wherein FLANK1 has the nucleotide G at position −4.

11. The dsODN molecule of claim 1 wherein FLANK1 is at least 6 nucleotides long.

12. The dsODN molecule of claim 1 wherein the FLANK1 is at least 7 nucleotides long.

13. The dsODN molecule of claim 1 in which the FLANK1-CORE-FLANK2 sequence is at least 14 nucleotides long.

14. The dsODN molecule of claim 1 in witch the FLANK1-CORE-FLANK2 sequence is at least 16 nucleotides long.

15. The dsODN molecule of claim 1 in which the FLANK1-CORE-FLANK2 sequence is 14 to 28 nucleotides long.

16. The dsODN molecule of claim 1 in which the FLANK1-CORE-FLANK2 sequence is 16 to 24 nucleotides long.

17. The dsODN molecule of claim 1, in which at least one of the sense and antisense strands has a modified backbone, comprising one or more phosphodiester linkages substituted by another linkage.

18. The dsODN molecule of claim 14, comprising one or more phosphodiester linkages substituted by a linkage selected from the group consisting of phosphothioate, phosphodithioate, and phosphoamidate linkages.

19. The dsODN molecule of claim 1, wherein FLANK1-CORE-FLANK2 is selected from the sequences listed in Tables 2A and 2B.

20. The dsODN molecule of claim 19 wherein FLANK1-CORE-FLANK2 is selected from the group of decoy sequence Nos. 893 (SEQ ID NO: 161), 895 (SEQ ID NO: 162), 985 (SEQ ID NO: 207), 987 (SEQ ID NO: 208), 963 (SEQ ID NO: 196), 993 (SEQ ID NO: 211), and 995 (SEQ ID NO: 212).

21. The dsODN molecule of claim 20 wherein FLANK1-CORE-FLANK2 is decoy sequence No. 895 (SEQ ID NO: 162).

22. The dsODN molecule of claim 20 wherein FLANK1-CORE-FLANK2 is decoy sequence No. 985 (SEQ ID NO: 207).

23. The dsODN molecule of claim 1 which is selected from the group of decoy sequence Nos. 893 (SEQ ID NO: 161), 895 (SEQ ID NO: 162), 985 (SEQ ID NO: 207), 987 (SEQ ID NO: 208), 963 (SEQ ID NO: 196), 993 (SEQ ID NO: 211), and 995 (SEQ ID NO: 212).

24. The dsODN molecule of claim 23 which is decoy sequence No. 895 (SEQ ID NO: 162).

25. The dsODN molecule of claim 23 which is decoy sequence No. 985 (SEQ ID NO: 207).

26. A method for modulating the transcription of a gene that is regulated by a HIF transcription factor, comprising introducing into the nucleus of a cell containing said gene a dsODN molecule according to any one of claims 1-25.

27. The method of claim 26 wherein said HIF transcription factor is HIF-1.

28. The method of claim 27 which is performed in vivo.

29. The method of claim 27 which is performed ex vivo.

30. The method of claim 27 wherein said HIF dsODN molecule is capable of episomal replication in said cell.

31. The method of claim 27 wherein said HIF dsODN molecule is delivered as a composition.

32. The method of claim 31 wherein said composition comprises liposomes, and said HIF dsODN is within the lumen of said liposomes.

33. The method of claim 32 wherein said liposomes comprise lipid and a viral coat protein.

34. The method of claim 27 wherein said HIF dsODN is introduced into the nucleus of said cell by pressure-mediated transfection.

35. A method for the prevention or treatment in a mammalian host of a disease or condition associated with HIF-regulated gene transcription, comprising introducing into the cells of said mammal in vivo or ex vivo an effective amount of a double-stranded HIF decoy oligodeoxynucleotide (dsODN) molecule comprising a core sequence that is capable of specific binding to a HIF transcription factor.

36. The method of claim 35 wherein said HIF transcription factor is HIF-1.

37. The method of claim 36 wherein said dsODN molecule is any one of the dsODN molecules of claims 1-25.

38. The method of claim 37 wherein said disease or condition is cancer.

39. The method of claim 38 wherein said cancer is selected from the group consisting of kidney, pancreatic, colon and lung cancer.

40. The method of claim 38 further comprising the administration of an additional anti-angiogen.

41. The method of claim 40 wherein said additional anti-angiogenic agent is selected from the group consisting of anti-EGF agents, anti-VEGF agents, matrix metalloproteinase inhibitors, vascular targeting agents, and integrin antagonists.

42. The method of claim 40 wherein said additional anti-angiogenic agent is selected from the group consisting of Avastin™ (bevacizumab, Genentech, Inc.); angiostatin; endostatin; Panzem® (2-methoxyestradiol, EntreMed, Inc.); Iressa® (gefitinib, AstraZeneca), and thalidomide.

43. The method of claim 37 wherein said disease or condition is an inflammatory disease.

44. The method of claim 37 wherein said disease or condition involves hypoxia in its pathology.

45. The method of claim 37 wherein said disease or condition is a cardiovascular disease or stroke.

46. The method of claim 37 wherein said disease or condition is selected from the group consisting of diabetic retinopathy, Age-related Macular Degeneration, and corneal neovascularization.

47. The method of claim 37 wherein said disease or condition is associated with pathogenic blood vessel growth.

48. The method of claim 37 wherein said disease or condition is a musculosceletal disorder.

49. A composition comprising a dsODN molecule according to any one of claims 1-25 and a carrier.

50. The composition of claim 49 wherein said carrier facilitates delivery in the nucleus of a cell.

51. The composition of claim 49 wherein said composition is a liposome composition.

52. The composition of claim 51 wherein said dsODN molecule is within the lumen of the liposome.

53. The method of claim 52 wherein said liposomes comprise lipid and a viral coat protein.


SEQUENCE LISTING (214 sequences)

SEQ ID NO: 1 - DNA, 8 bp, Artificial Sequence
  HIF-1 binding sites and flanking sequences
bacgtgcn

SEQ ID NO: 2 - DNA, 52 bp, Artificial Sequence
  HIF-1 binding sites and flanking sequences
gtgtgctcccagtcagtcaatcctcacgtttatgatggatgaatgaaggcag

SEQ ID NO: 3 - DNA, 49 bp, Artificial Sequence
  HIF-1 binding sites and flanking sequences
ttgtgttattagtcaccaacaggcaacgtgcagccggagataaggccag

SEQ ID NO: 4 - DNA, 56 bp, Artificial Sequence
  HIF-1 binding sites and flanking sequences
atccccccgcccacagagaggacgtgccacgccagcacgtccgctctccttgccag

SEQ ID NO: 5 - DNA, 57 bp, Artificial Sequence
  HIF-1 binding sites and flanking sequences
tgtgctcccagtcagtcaatcctcacgtttatgatggatgaatgaaggcagtcaggt

SEQ ID NO: 6 - DNA, 62 bp, Artificial Sequence
  HIF-1 binding sites and flanking sequences
gtgatgaaagagcacaaacgggtgacaaacgtgtctagcgtgattcatcatgaacaggcaca

SEQ ID NO: 7 - DNA, 57 bp, Artificial Sequence
  HIF-1 binding sites and flanking sequences
tgcttggtaaactgtaaaatgattagcatacgtgaagcgttagtgtgctccctggca

SEQ ID NO: 8 - DNA, 62 bp, Artificial Sequence
  HIF-1 binding sites and flanking sequences
gagcgagccgctgggtgcaggcaggcgacgtgctgccgggctaggctgcccgggggagatga

SEQ ID NO: 9 - DNA, 25 bp, Artificial Sequence
  HIF-1 binding sites and flanking sequences
gtggtccgagtcacgtccgaggggg

SEQ ID NO: 10 - DNA, 26 bp, Artificial Sequence
  HIF-1 binding sites and flanking sequences
cttcacgtgcggggaccagggaccgt

SEQ ID NO: 11 - DNA, 26 bp, Artificial Sequence
  HIF-1 binding sites and flanking sequences
cgcaggcgcaggcggcgcacgtggcc

SEQ ID NO: 12 - DNA, 50 bp, Artificial Sequence
  HIF-1 binding sites and flanking sequences
gagtgcgtgcgggactcggagtacgtgacggagccccgagctctcatgcc

SEQ ID NO: 13 - DNA, 53 bp, Artificial Sequence
  HIF-1 binding sites and flanking sequences
ggggccccagagcgacgctgagtgcgtgcgggactcggagtacgtgacggagc

SEQ ID NO: 14 - DNA, 52 bp, Artificial Sequence
  HIF-1 binding sites and flanking sequences
gcaaggtcgagggccggacgtggggccccagagcgacgctgagtgcgtgcgg

SEQ ID NO: 15 - DNA, 52 bp, Artificial Sequence
  HIF-1 binding sites and flanking sequences
ggggcgtgagcggggctgctgcagacgtgcgtgtgggtcatgggggctgctc

SEQ ID NO: 16 - DNA, 43 bp, Artificial Sequence
  HIF-1 binding sites and flanking sequences
gccctacgtgctgtctcacacagcctgtctgacctctcgacct

SEQ ID NO: 17 - DNA, 20 bp, Artificial Sequence
  HIF-1 binding sites and flanking sequences
ctccggctgcacgttgcctg

SEQ ID NO: 18 - DNA, 55 bp, Artificial Sequence
  HIF-1 binding sites and flanking sequences
atggagacataattgaggaacaacgtggaattagtgtcatagcaaatgatctagg

SEQ ID NO: 19 - DNA, 31 bp, Artificial Sequence
  HIF-1 binding sites and flanking sequences
gagcggacgtgctggcgtggcacgtcctctc

SEQ ID NO: 20 - DNA, 51 bp, Artificial Sequence
  HIF-1 binding sites and flanking sequences
gacgcccgcccccggcccagcctacacgtgggttcccgcacgtccgctggg

SEQ ID NO: 21 - DNA, 51 bp, Artificial Sequence
  HIF-1 binding sites and flanking sequences
cgtcagagtgggagcccagcggacgtgcgggaacccacgtgtaggctgggc

SEQ ID NO: 22 - DNA, 30 bp, Artificial Sequence
  HIF-1 binding sites and flanking sequences
gtgactacgtgctgcctaggggccactgcc

SEQ ID NO: 23 - DNA, 37 bp, Artificial Sequence
  HIF-1 binding sites and flanking sequences
cctgaatgctcttacacacgtacacacacagagcagc

SEQ ID NO: 24 - DNA, 26 bp, Artificial Sequence
  HIF-1 binding sites and flanking sequences
ccgggtagctggcgtacgtgctgcag

SEQ ID NO: 25 - DNA, 54 bp, Artificial Sequence
  Gene PGK1_1; HIF-1 binding sites and flanking sequences
ccttgcggttcgcggcgtgccggacgtgacaaacggaagccgcacgtctcacta

SEQ ID NO: 26 - DNA, 54 bp, Artificial Sequence; identical to SEQ ID NO: 25
  Gene PGK1_2; HIF-1 binding sites and flanking sequences

SEQ ID NO: 27 - DNA, 20 bp, Artificial Sequence
  HIF-1 binding sites and flanking sequences
ctgccagtgcacgtcagtgg

SEQ ID NO: 28 - DNA, 72 bp, Artificial Sequence
  HIF-1 binding sites and flanking sequences
gcccggccgctgtcaccgggcaggagagaacgttgcttacgtgcgcccggagtccattggccaaggcgggcc

SEQ ID NO: 29 - DNA, 51 bp, Artificial Sequence
  HIF-1 binding sites and flanking sequences
aaggccctgggtccacaggcgtgccgtctgacacgcatcaggcaggcactc

SEQ ID NO: 30 - DNA, 53 bp, Artificial Sequence
  HIF-1 binding sites and flanking sequences
ccatttctagggccttgggtccacaggcgtgctggctgacacgcatcaggccg

SEQ ID NO: 31 - DNA, 33 bp, Artificial Sequence
  HIF-1 binding sites and flanking sequences
ttcctgcacgtacacacaaagcgcacgtatttc

SEQ ID NO: 32 - DNA, 61 bp, Artificial Sequence
  HIF-1 binding sites and flanking sequences
tcagagcacctcgcgagcgtacgtgcctcaggaagtgacgcacagcccccctgggggccgg

SEQ ID NO: 33 - DNA, 65 bp, Artificial Sequence
  HIF-1 binding sites and flanking sequences
gggttttgccagactccacagtgcatacgtgggctccaacaggtcctcttccctcccagtcactg

SEQ ID NO: 34 - DNA, 18 bp, Artificial Sequence
  initial HIF-1 decoy
gccctacgtgctgtctca

SEQ ID NO: 35 - DNA, 18 bp, Artificial Sequence
  initial HIF-1 decoy
tgagacagcacgtagggc

SEQ ID NO: 36 - DNA, 18 bp, Artificial Sequence
  initial HIF-1 decoy
ctgtcctccgactgcatg

SEQ ID NO: 37 - DNA, 18 bp, Artificial Sequence
  initial HIF-1 decoy
catgcagtcggaggacag

SEQ ID NO: 38 - DNA, 25 bp, Artificial Sequence
  initial HIF-1 decoy
ccccctcggacgtgactcggaccac

SEQ ID NO: 39 - DNA, 25 bp, Artificial Sequence
  initial HIF-1 decoy
gtggtccgagtcacgtccgaggggg

SEQ ID NO: 40 - DNA, 24 bp, Artificial Sequence
  initial HIF-1 decoy
tctgtacgtgaccacactcacctc

SEQ ID NO: 41 - DNA, 24 bp, Artificial Sequence
  initial HIF-1 decoy
gaggtgagtgtggtcacgtacaga

SEQ ID NO: 42 - DNA, 20 bp, Artificial Sequence
  initial HIF-1 decoy
agggccggacgtggggcccc

SEQ ID NO: 43 - DNA, 20 bp, Artificial Sequence
  initial HIF-1 decoy
ggggccccacgtccggccct

SEQ ID NO: 44 - DNA, 20 bp, Artificial Sequence
  initial HIF-1 decoy
acgctgagtgcgtgcgggac

SEQ ID NO: 45 - DNA, 20 bp, Artificial Sequence
  initial HIF-1 decoy
gtcccgcacgcactcagcgt

SEQ ID NO: 46 - DNA, 24 bp, Artificial Sequence
  initial HIF-1 decoy
gccctacgtgctgtctcacacagc

SEQ ID NO: 47 - DNA, 24 bp, Artificial Sequence
  initial HIF-1 decoy
gctgtgtgagacagcacgtagggc

SEQ ID NO: 48 - DNA, 23 bp, Artificial Sequence
  initial HIF-1 decoy
gtgagacgtgcggcttccgtttg

SEQ ID NO: 49 - DNA, 23 bp, Artificial Sequence
  initial HIF-1 decoy
caaacggaagccgcacgtctcac

SEQ ID NO: 50 - DNA, 21 bp, Artificial Sequence
  initial HIF-1 decoy
ctgccgacgtgcgctccggag

SEQ ID NO: 51 - DNA, 21 bp, Artificial Sequence
  initial HIF-1 decoy
ctccggagcgcacgtcggcag

SEQ ID NO: 52 - DNA, 33 bp, Artificial Sequence
  initial HIF-1 decoy
gaaatacgtgcgctttgtgtgtacgtgcaggaa

SEQ ID NO: 53 - DNA, 33 bp, Artificial Sequence; identical to SEQ ID NO: 31
  initial HIF-1 decoy

SEQ ID NO: 54 - DNA, 21 bp, Artificial Sequence
  initial HIF-1 decoy
cgcgagcgtacgtgcctcagg

SEQ ID NO: 55 - DNA, 21 bp, Artificial Sequence
  initial HIF-1 decoy
cctgaggcacgtacgctcgcg

SEQ ID NO: 56 - DNA, 21 bp, Artificial Sequence
  initial HIF-1 decoy
tgcatacgtgggctccaacag

SEQ ID NO: 57 - DNA, 21 bp, Artificial Sequence
  initial HIF-1 decoy
ctgttggagcccacgtatgca

SEQ ID NO: 58 - DNA, 16 bp, Artificial Sequence
  initial HIF-1 decoy
aggagacgtgcgagaa

SEQ ID NO: 59 - DNA, 16 bp, Artificial Sequence
  initial HIF-1 decoy
ttctcgcacgtctcct

SEQ ID NO: 60 - DNA, 16 bp, Artificial Sequence
  initial HIF-1 decoy
aggttacgtgcggaca

SEQ ID NO: 61 - DNA, 16 bp, Artificial Sequence
  initial HIF-1 decoy
tgtccgcacgtaacct

SEQ ID NO: 62 - DNA, 16 bp, Artificial Sequence
  initial HIF-1 decoy
aggagacgtgctgcct

SEQ ID NO: 63 - DNA, 16 bp, Artificial Sequence
  initial HIF-1 decoy
aggcagcacgtctcct

SEQ ID NO: 64 - DNA, 18 bp, Artificial Sequence
  initial HIF-1 decoy
tccaatacgtgcagtact

SEQ ID NO: 65 - DNA, 18 bp, Artificial Sequence
  initial HIF-1 decoy
agtactgcacgtattgga

SEQ ID NO: 66 - DNA, 18 bp, Artificial Sequence
  initial HIF-1 decoy
tccaatgcgtgcagtact

SEQ ID NO: 67 - DNA, 18 bp, Artificial Sequence
  initial HIF-1 decoy
agtactgcacgcattgga

SEQ ID NO: 68 - DNA, 18 bp, Artificial Sequence
  initial HIF-1 decoy
ggccagacgtgccaccgg

SEQ ID NO: 69 - DNA, 18 bp, Artificial Sequence
  initial HIF-1 decoy
ccggtggcacgtctggcc

SEQ ID NO: 70 - DNA, 16 bp, Artificial Sequence
  initial HIF-1 decoy
aggcaacgtgcagccg

SEQ ID NO: 71 - DNA, 16 bp, Artificial Sequence
  initial HIF-1 decoy
cggctgcacgttgcct

SEQ ID NO: 72 - DNA, 16 bp, Artificial Sequence
  initial HIF-1 decoy
aggcaatacgcagccg

SEQ ID NO: 73 - DNA, 16 bp, Artificial Sequence
  initial HIF-1 decoy
cggctgcgtattgcct

SEQ ID NO: 74 - DNA, 29 bp, Artificial Sequence
  initial HIF-1 decoy
agcggacgtgcagaagttgcacgtcctct

SEQ ID NO: 75 - DNA, 29 bp, Artificial Sequence
  initial HIF-1 decoy
agaggacgtgcaacttctgcacgtccgct

SEQ ID NO: 76 - DNA, 18 bp, Artificial Sequence
  initial HIF-1 decoy
gtgcatacgtgggctcca

SEQ ID NO: 77 - DNA, 18 bp, Artificial Sequence
  initial HIF-1 decoy
tggagcccacgtatgcac

SEQ ID NO: 78 - DNA, 18 bp, Artificial Sequence
  initial HIF-1 decoy
gagcgtacgtgcctcagg

SEQ ID NO: 79 - DNA, 18 bp, Artificial Sequence
  initial HIF-1 decoy
cctgaggcacgtacgctc

SEQ ID NO: 80 - DNA, 18 bp, Artificial Sequence
  initial HIF-1 decoy
ggaacaacgtggaattag

SEQ ID NO: 81 - DNA, 18 bp, Artificial Sequence
  initial HIF-1 decoy
ctaattccacgttgttcc

SEQ ID NO: 82 - DNA, 18 bp, Artificial Sequence
  initial HIF-1 decoy
gcctacacgtgggttccc

SEQ ID NO: 83 - DNA, 18 bp, Artificial Sequence
  initial HIF-1 decoy
gggaacccacgtgtaggc

SEQ ID NO: 84 - DNA, 18 bp, Artificial Sequence
  initial HIF-1 decoy
cggagtacgtgacggagc

SEQ ID NO: 85 - DNA, 18 bp, Artificial Sequence
  initial HIF-1 decoy
gctccgtcacgtactccg

SEQ ID NO: 86 - DNA, 18 bp, Artificial Sequence
  initial HIF-1 decoy
ttgcttacgtgcgcccgg

SEQ ID NO: 87 - DNA, 18 bp, Artificial Sequence
  initial HIF-1 decoy
ccgggcgcacgtaagcaa

SEQ ID NO: 88 - DNA, 18 bp, Artificial Sequence
  initial HIF-1 decoy
gtgtgtacgtgcaggaaa

SEQ ID NO: 89 - DNA, 18 bp, Artificial Sequence
  initial HIF-1 decoy
tttcctgcacgtacacac

SEQ ID NO: 90 - DNA, 27 bp, Artificial Sequence
  initial HIF-1 decoy
gcggacgtgcgggaacccacgtgtagg

SEQ ID NO: 91 - DNA, 27 bp, Artificial Sequence
  initial HIF-1 decoy
cctacacgtgggttcccgcacgtccgc

SEQ ID NO: 92 - DNA, 16 bp, Artificial Sequence
  initial HIF-1 decoy
accgtacgtgctgatc

SEQ ID NO: 93 - DNA, 16 bp, Artificial Sequence
  initial HIF-1 decoy
gatcagcacgtacggt

SEQ ID NO: 94 - DNA, 16 bp, Artificial Sequence
  initial HIF-1 decoy
ctaatacgtgccgctg

SEQ ID NO: 95 - DNA, 16 bp, Artificial Sequence
  initial HIF-1 decoy
cagcggcacgtattag

SEQ ID NO: 96 - DNA, 16 bp, Artificial Sequence
  initial HIF-1 decoy
agcagacgtgcaggat

SEQ ID NO: 97 - DNA, 16 bp, Artificial Sequence
  initial HIF-1 decoy
atcctgcacgtctgct

SEQ ID NO: 98 - DNA, 16 bp, Artificial Sequence
  initial HIF-1 decoy
agcagacgtgcaggca

SEQ ID NO: 99 - DNA, 16 bp, Artificial Sequence
  initial HIF-1 decoy
tgcctgcacgtctgct

SEQ ID NO: 100 - DNA, 16 bp, Artificial Sequence
  initial HIF-1 decoy
tccgtacgtgctgcac

SEQ ID NO: 101 - DNA, 16 bp, Artificial Sequence
  initial HIF-1 decoy
gtgcagcacgtacgga

SEQ ID NO: 102 - DNA, 16 bp, Artificial Sequence
  initial HIF-1 decoy
agcagacgtgcagggt

SEQ ID NO: 103 - DNA, 16 bp, Artificial Sequence
  initial HIF-1 decoy
accctgcacgtctgct

SEQ ID NO: 104 - DNA, 16 bp, Artificial Sequence
  initial HIF-1 decoy
accgtacgtgctgcca

SEQ ID NO: 105 - DNA, 16 bp, Artificial Sequence
  initial HIF-1 decoy
tggcagcacgtacggt

SEQ ID NO: 106 - DNA, 16 bp, Artificial Sequence
  initial HIF-1 decoy
tccgtacgtgctgcgt

SEQ ID NO: 107 - DNA, 16 bp, Artificial Sequence
  initial HIF-1 decoy
acgcagcacgtacgga

SEQ ID NO: 108 - DNA, 16 bp, Artificial Sequence
  initial HIF-1 decoy
tgcagacgtgcaggtc

SEQ ID NO: 109 - DNA, 16 bp, Artificial Sequence
  initial HIF-1 decoys
gacctgcacgtctgca

SEQ ID NO: 110 - DNA, 16 bp, Artificial Sequence
  initial HIF-1 decoys
accgtacgtgctgcta

SEQ ID NO: 111 - DNA, 16 bp, Artificial Sequence
  initial HIF-1 decoys
tagcagcacgtacggt

SEQ ID NO: 112 - DNA, 22 bp, Artificial Sequence
  initial HIF-1 decoys
ggctgctgcagacgtgcaggtc

SEQ ID NO: 113 - DNA, 22 bp, Artificial Sequence
  initial HIF-1 decoys
gacctgcacgtctgcagcagcc

SEQ ID NO: 114 - DNA, 21 bp, Artificial Sequence
  initial HIF-1 decoys
ggctgcaggagacgtggagaa

SEQ ID NO: 115 - DNA, 21 bp, Artificial Sequence
  initial HIF-1 decoys
ttctccacgtctcctgcagcc

SEQ ID NO: 116 - DNA, 16 bp, Artificial Sequence
  initial HIF-1 decoys
agaagacgtgcaggat

SEQ ID NO: 117 - DNA, 16 bp, Artificial Sequence
  initial HIF-1 decoys
atcctgcacgtcttct

SEQ ID NO: 118 - DNA, 16 bp, Artificial Sequence
  initial HIF-1 decoys
tacagacgtgcaggtc

SEQ ID NO: 119 - DNA, 16 bp, Artificial Sequence
  initial HIF-1 decoys
gacctgcacgtctgta

SEQ ID NO: 120 - DNA, 22 bp, Artificial Sequence
  initial HIF-1 decoys
ggctgcaccgtacgtgctgatc

SEQ ID NO: 121 - DNA, 22 bp, Artificial Sequence
  initial HIF-1 decoys
gatcagcacgtacggtgcagcc

SEQ ID NO: 122 - DNA, 16 bp, Artificial Sequence
  initial HIF-1 decoys
tgcatacgtgcaggtc

SEQ ID NO: 123 - DNA, 16 bp, Artificial Sequence
  initial HIF-1 decoys
gacctgcacgtatgca

SEQ ID NO: 124 - DNA, 22 bp, Artificial Sequence
  initial HIF-1 decoys
ggctgctgcatacgtgcaggtc

SEQ ID NO: 125 - DNA, 22 bp, Artificial Sequence
  initial HIF-1 decoys
gacctgcacgtatgcagcagcc

SEQ ID NO: 126 - DNA, 5 bp, Artificial Sequence
  HRE response element
acgtg

SEQ ID NO: 127 - DNA, 4 bp, Artificial Sequence
  flanking sequence
gcgt

SEQ ID NO: 128 - DNA, 18 bp, Artificial Sequence
  dsODN sense strand
gccctacgtgctgtctca

SEQ ID NO: 129 - DNA, 18 bp, Artificial Sequence
  dsODN antisense strand
tgagacagcacgtagggc

SEQ ID NO: 130 - DNA, 21 bp, Artificial Sequence
  HIF-1 decoy
caccagcgtacgtgcctcagg

SEQ ID NO: 131 - DNA, 21 bp, Artificial Sequence
  HIF-1 binding site from Transferrin receptor
cgcgagcgtacgtgcctcagg

SEQ ID NO: 132 - DNA, 18 bp, Artificial Sequence
  HIF-1binding site from Erythropoietin promoter
gccctacgtgctgtctca

SEQ ID NO: 133 - DNA, 18 bp, Artificial Sequence
  Oct-1 binding site
gccctacgtgctgtctca

SEQ ID NO: 134 - DNA, 4 bp, Artificial Sequence
  HIF-1 core binding sequence
cgtg

SEQ ID NO: 135 - DNA, 21 bp, Artificial Sequence
  initial HIF-1 decoys
cctgaggcacgtacgctggtg

SEQ ID NO: 136 - DNA, 59 bp, Artificial Sequence
  HIF-1 binding sites and flanking sequences
gtctgccgcttcgggcggggcggctcgcgtgctggggtggggctttccttctctagtct

SEQ ID NO: 137 - DNA, 59 bp, Artificial Sequence
  HIF-1 binding sites and flanking sequences
agatcagaataggtaaccaagacttagcgtgttcctcctttcttgcgctatagagttgt

SEQ ID NO: 138 - DNA, 59 bp, Artificial Sequence
  HIF-1 binding sites and flanking sequences
ctgtctcctgagtgttgggattaaaggcgtgtgccgtcaccacctggctcactaccaca

SEQ ID NO: 139 - DNA, 59 bp, Artificial Sequence
  HIF-1 binding sites and flanking sequences
cctcagagtccttccccaggaagttcgcgtgacgaagataaaacaatattcaaatgtgg

SEQ ID NO: 140 - DNA, 59 bp, Artificial Sequence
  HIF-1 binding sites and flanking sequences
tgcactaatgcacacccaccactaatgcatgccccatcactaatgcacacccccatcac

SEQ ID NO: 141 - DNA, 59 bp, Artificial Sequence
  HIF-1 binding sites and flanking sequences
ccggaccggtcacgtgggcgcggccggcgtgcgcggggcggggcggagcggggcctggc

SEQ ID NO: 142 - DNA, 59 bp, Artificial Sequence
  HIF-1 binding sites and flanking sequences
cacccacggctcccctcccccagctagcgtgacagcactgggacccgcgcccggtagtg

SEQ ID NO: 143 - DNA, 59 bp, Artificial Sequence
  HIF-1 binding sites and flanking sequences
ggctggggacaaggtgccccggagttgcgtgagaagagcctggaggcctgcgcagccac

SEQ ID NO: 144 - DNA, 59 bp, Artificial Sequence
  HIF-1 binding sites and flanking sequences
ggctggggacaaggtgccccggagttgcgtgagaagagcctggaggcccgcgcagccac

SEQ ID NO: 145 - DNA, 59 bp, Artificial Sequence
  HIF-1 binding sites and flanking sequences
ggcacgcacacaggttcctgaggctagcgtgcgtaagcctgctccatcctctgggggca

SEQ ID NO: 146 - DNA, 59 bp, Artificial Sequence
  HIF-1 binding sites and flanking sequences
agccccagaccccctcctgctgtctcgcgtggatccttcctccaccctttcctccacca

SEQ ID NO: 147 - DNA, 59 bp, Artificial Sequence
  HIF-1 binding sites and flanking sequences
ttccctggattactgagtccaagctcgcgtgagaagcgcaacgaccccagcccagaggt

SEQ ID NO: 148 - DNA, 59 bp, Artificial Sequence
  HIF-1 binding sites and flanking sequences
catttttgcacccactggaacgctgggcgtgcagatgcctccccagcgctacagcctac

SEQ ID NO: 149 - DNA, 59 bp, Artificial Sequence
  HIF-1 binding sites and flanking sequences
ttcccggggttactgagtcccggctcgcgtgagaagcgctgcgaccccagccctgaggt

SEQ ID NO: 150 - DNA, 59 bp, Artificial Sequence
  HIF-1 binding sites and flanking sequences
gcccccaccctacccccggctagcttgcgtgcgccggcgacatccctctagggggcaga

SEQ ID NO: 151 - DNA, 59 bp, Artificial Sequence
  HIF-1 binding sites and flanking sequences
gcccggaggcgcggtgcccaggcgttgcgtgagaaggaccggaggcccgcgcagccacc

SEQ ID NO: 152 - DNA, 59 bp, Artificial Sequence
  HIF-1 binding sites and flanking sequences
gccccgccccgactccctcccccctcgcgtgactgcgagcccccgcgccgggccgggga

SEQ ID NO: 153 - DNA, 59 bp, Artificial Sequence
  HIF-1 binding sites and flanking sequences
tacgcagcagggcaggagattggggggcgtggcacactctggagcaccttgcctcccca

SEQ ID NO: 154 - DNA, 59 bp, Artificial Sequence
  HIF-1 binding sites and flanking sequences
gtatgtgtgtgtgagcatgtttatgtgcgtgtgtatatgagtgtgtgtgtgtgcatgta

SEQ ID NO: 155 - DNA, 59 bp, Artificial Sequence
  HIF-1 binding sites and flanking sequences
ggagcccagcggacgtgcgggaacccacgtgtaggctgggccgggggcgggcgtccagc

SEQ ID NO: 156 - DNA, 59 bp, Artificial Sequence
  HIF-1 binding sites and flanking sequences
tccagcctggcttccgattctgccttgcgtgtttgtgacgagccagcgagccgggacgt

SEQ ID NO: 157 - DNA, 59 bp, Artificial Sequence
  HIF-1 binding sites and flanking sequences
aggcaagtgggcttcttttgagagttgcgtgcccctttccgtccagcctggcttccgat

SEQ ID NO: 158 - DNA, 59 bp, Artificial Sequence
  HIF-1 binding sites and flanking sequences
gtctttgatcaagatccttttctgctgcgtgtggttaaaacatacaacaaagtcaaaag

SEQ ID NO: 159 - DNA, 60 bp, Artificial Sequence
  HIF-1 binding sites and flanking sequences
aaacttgtgggtgggaacaggaactcgcgtgccagccaggtgtgcatgactagctctggg

SEQ ID NO: 160 - DNA, 59 bp, Artificial Sequence
  HIF-1 binding sites and flanking sequences
gggctctgccagactccacagtgcatacgtgggcttccacaggtcgtctccctccgggc

SEQ ID NO: 161 - DNA, 21 bp, Artificial Sequence
  initial HIF-1 decoy
cacgagcgtacgtgcctcagg

SEQ ID NO: 162 - DNA, 21 bp, Artificial Sequence
  initial HIF-1 decoy
caccagcgtacgtgcctcagg

SEQ ID NO: 163 - DNA, 19 bp, Artificial Sequence
  initial HIF-1 decoy
ccagcgtacgtgcctcagg

SEQ ID NO: 164 - DNA, 19 bp, Artificial Sequence
  initial HIF-1 decoy
cgagcgtacgtgcctcagg

SEQ ID NO: 165 - DNA, 21 bp, Artificial Sequence
  initial HIF-1 decoy
cacgtgcatacgtgggctcca

SEQ ID NO: 166 - DNA, 21 bp, Artificial Sequence
  initial HIF-1 decoy
cacctgcatacgtgggctcca

SEQ ID NO: 167 - DNA, 26 bp, Artificial Sequence
  initial HIF-1 decoy
gatcgccctacgtgctgtctcagatc

SEQ ID NO: 168 - DNA, 26 bp, Artificial Sequence
  initial HIF-1 decoy
gaaatacgtgcgtgtgtacgtgcagg

SEQ ID NO: 169 - DNA, 26 bp, Artificial Sequence
  initial HIF-1 decoy
agcggacgtgcagatgcacgtcctct

SEQ ID NO: 170 - DNA, 21 bp, Artificial Sequence
  initial HIF-1 decoy
cacagcgtacgtgctgtctca

SEQ ID NO: 171 - DNA, 21 bp, Artificial Sequence
  initial HIF-1 decoy
caggctccgactacggctgac

SEQ ID NO: 172 - DNA, 21 bp, Artificial Sequence
  initial HIF-1 decoy
agatccgacgtaccgaccaag

SEQ ID NO: 173 - DNA, 21 bp, Artificial Sequence
  initial HIF-1 decoy
ctaagcgagtagcgagtagcc

SEQ ID NO: 174 - DNA, 21 bp, Artificial Sequence
  initial HIF-1 decoy
cgctacgagctctactccagg

SEQ ID NO: 175 - DNA, 21 bp, Artificial Sequence
  initial HIF-1 decoy
cgctcgacgagctctactcca

SEQ ID NO: 176 - DNA, 21 bp, Artificial Sequence
  initial HIF-1 decoy
caccagcgtaaaagcctcagg

SEQ ID NO: 177 - DNA, 19 bp, Artificial Sequence
  initial HIF-1 decoy
ccagcgtacgtgcctcagg

SEQ ID NO: 178 - DNA, 19 bp, Artificial Sequence
  initial HIF-1 decoy
ccagcgttcgtgcctcagg

SEQ ID NO: 179 - DNA, 19 bp, Artificial Sequence
  initial HIF-1 decoy
ccagcgtgcgtgcctcagg

SEQ ID NO: 180 - DNA, 19 bp, Artificial Sequence
  initial HIF-1 decoy
ccagcgtccgtgcctcagg

SEQ ID NO: 181 - DNA, 19 bp, Artificial Sequence
  initial HIF-1 decoy
ccagcgtatgtgcctcagg

SEQ ID NO: 182 - DNA, 19 bp, Artificial Sequence
  initial HIF-1 decoy
ccagcgtaagtgcctcagg

SEQ ID NO: 183 - DNA, 19 bp, Artificial Sequence
  initial HIF-1 decoy
ccagcgtaggtgcctcagg

SEQ ID NO: 184 - DNA, 19 bp, Artificial Sequence
  initial HIF-1 decoy
ccagcgtacatgcctcagg

SEQ ID NO: 185 - DNA, 19 bp, Artificial Sequence
  initial HIF-1 decoy
ccagcgtacctgcctcagg

SEQ ID NO: 186 - DNA, 19 bp, Artificial Sequence
  initial HIF-1 decoy
ccagcgtacttgcctcagg

SEQ ID NO: 187 - DNA, 19 bp, Artificial Sequence
  initial HIF-1 decoy
ccagcgtacgagcctcagg

SEQ ID NO: 188 - DNA, 19 bp, Artificial Sequence
  initial HIF-1 decoy
ccagcgtacgcgcctcagg

SEQ ID NO: 189 - DNA, 19 bp, Artificial Sequence
  initial HIF-1 decoy
ccagcgtacgggcctcagg

SEQ ID NO: 190 - DNA, 19 bp, Artificial Sequence
  initial HIF-1 decoy
ccagcgtacgtccctcagg

SEQ ID NO: 191 - DNA, 19 bp, Artificial Sequence
  initial HIF-1 decoy
ccagcgtacgtacctcagg

SEQ ID NO: 192 - DNA, 19 bp, Artificial Sequence
  initial HIF-1 decoy
ccagcgtacgttcctcagg

SEQ ID NO: 193 - DNA, 19 bp, Artificial Sequence
  initial HIF-1 decoy
ccagcgcacgtgcctcagg

SEQ ID NO: 194 - DNA, 19 bp, Artificial Sequence
  initial HIF-1 decoy
ccagcgaacgtgcctcagg

SEQ ID NO: 195 - DNA, 19 bp, Artificial Sequence
  initial HIF-1 decoy
ccagcggacgtgcctcagg

SEQ ID NO: 196 - DNA, 19 bp, Artificial Sequence
  initial HIF-1 decoy
ccagcctacgtgcctcagg

SEQ ID NO: 197 - DNA, 19 bp, Artificial Sequence
  initial HIF-1 decoy
ccagcttacgtgcctcagg

SEQ ID NO: 198 - DNA, 19 bp, Artificial Sequence
  initial HIF-1 decoy
ccagcatacgtgcctcagg

SEQ ID NO: 199 - DNA, 19 bp, Artificial Sequence
  initial HIF-1 decoy
ccagggtacgtgcctcagg

SEQ ID NO: 200 - DNA, 19 bp, Artificial Sequence
  initial HIF-1 decoy
ccagtgtacgtgcctcagg

SEQ ID NO: 201 - DNA, 19 bp, Artificial Sequence
  initial HIF-1 decoy
ccagagtacgtgcctcagg

SEQ ID NO: 202 - DNA, 19 bp, Artificial Sequence
  initial HIF-1 decoy
ccaccgtacgtgcctcagg

SEQ ID NO: 203 - DNA, 19 bp, Artificial Sequence
  initial HIF-1 decoy
ccaacgtacgtgcctcagg

SEQ ID NO: 204 - DNA, 19 bp, Artificial Sequence
  initial HIF-1 decoy
ccatcgtacgtgcctcagg

SEQ ID NO: 205 - DNA, 19 bp, Artificial Sequence
  initial HIF-1 decoy
ccagcgtacgtggctcagg

SEQ ID NO: 206 - DNA, 19 bp, Artificial Sequence
  initial HIF-1 decoy
ccagcgtacgtgtctcagg

SEQ ID NO: 207 - DNA, 19 bp, Artificial Sequence
  initial HIF-1 decoy
ccagcgtacgtgactcagg

SEQ ID NO: 208 - DNA, 19 bp, Artificial Sequence
  initial HIF-1 decoy
ccagcgtacgtgcgtcagg

SEQ ID NO: 209 - DNA, 19 bp, Artificial Sequence
  initial HIF-1 decoy
ccagcgtacgtgcttcagg

SEQ ID NO: 210 - DNA, 19 bp, Artificial Sequence
  initial HIF-1 decoy
ccagcgtacgtgcatcagg

SEQ ID NO: 211 - DNA, 19 bp, Artificial Sequence
  initial HIF-1 decoy
ccagcgtacgtgccacagg

SEQ ID NO: 212 - DNA, 19 bp, Artificial Sequence
  initial HIF-1 decoy
ccagcgtacgtgccgcagg

SEQ ID NO: 213 - DNA, 19 bp, Artificial Sequence
  initial HIF-1 decoy
ccggggcacgtgccccagg

SEQ ID NO: 214 - DNA, 19 bp, Artificial Sequence
  initial HIF-1 decoy
ccatggcacgtgcctcagg